(19) United States
(12) Patent Application Publication
(10) Pub. No.: US 2014/0309226 A1
(43) Pub. Date: Oct. 16, 2014

(54) PIPERIDINYL-SUBSTITUTED CYCLIC UREAS AS GPR119 MODULATORS

(71) Applicant: Array BioPharma Inc., Boulder, CO (US)

(72) Inventors: Jay Bradford Fell, Boulder, CO (US); John P. Fischer, Boulder, CO (US); Ronald J. Hinklin, Boulder, CO (US)

(21) Appl. No.: 14/359,036

(22) PCT Filed: Nov. 14, 2012
(86) PCT No.: PCT/US2012/065036

§ 371 Date: May 16, 2014

Related U.S. Application Data
(60) Provisional application No. 61/560,433, filed on Nov. 16, 2011.

(51) Int. Cl. C07D 417/14; C07D 401/14; C07D 413/14

(52) U.S. Cl. CPC C07D 417/14; C07D 413/14; C07D 401/14
USPC 514/235.8; 546/209; 514/326; 544/316; 514/274; 546/194; 514/318; 544/405; 514/255.05; 544/295; 544/130

(57) ABSTRACT

Compounds of Formula I: and pharmaceutically acceptable salts thereof, in which X¹, X², R³, R⁴, R⁵, R^{x}, R⁷, R⁹, R¹⁰ and n have the meanings given in the specification, are modulators of GPR119 and are useful in the treatment or prevention of diseases such as such as, but not limited to, type 2 diabetes, diabetic complications, symptoms of diabetes, metabolic syndrome, obesity, dyslipidemia, and related conditions.

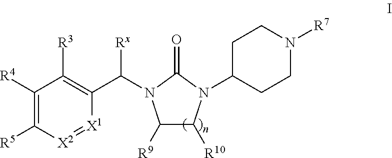

The present invention relates to novel compounds, to pharmaceutical compositions comprising the compounds, to processes for making the compounds, and to the use of the compounds in therapy. More particularly, it relates to certain piperidinyl-substituted ureas which are modulators of GPR¹¹⁹ and are useful in the treatment or prevention of diseases such as, but not limited to, type 2 diabetes, diabetic complications, symptoms of diabetes, metabolic syndrome, obesity, dyslipidemia, and related conditions. In addition, the compounds are useful in decreasing food intake, decreasing weight gain, and increasing satiety in mammals.

Diabetes is diagnosed by elevated fasting plasma glucose levels ≧126 mg/dL or by plasma glucose levels after an oral glucose tolerance test ≧200 mg/dL. Diabetes is associated with the classic symptoms of polydipsia, polyphagia and polyuria (The Expert Committee on the Diagnosis and Classification of Diabetes Mellitus, Diabetes Care, 1998, 21, S5-19). Of the two major forms of diabetes, insulin dependent diabetes mellitus (Type I) accounts for 5-10% of the diabetic population. Type I diabetes is characterized by near total beta cell loss in the pancreas and little or no circulating insulin. Non-insulin dependent diabetes mellitus (Type 2 diabetes) is the more common form of diabetes. Type 2 diabetes is a chronic metabolic disease that develops from a combination of insulin resistance in the muscle, fat, and liver and from partial beta cell loss in the pancreas. The disease progresses with the inability of the pancreas to secrete sufficient insulin to overcome such resistance. Uncontrolled type 2 diabetes is associated with an increased risk of heart disease, stroke, neuropathy, retinopathy and nephropathy among other diseases.

Obesity is a medical condition characterized by high levels of adipose tissue in the body. Body mass index is calculated by dividing weight by height squared (BMI=kg/m²), where a person with a BMI of ≧30 is considered obese and medical intervention is recommended (For the Clinical Efficacy Assessment Subcommittee of the American College of Physicians. Pharmacological and surgical management of obesity in primary care: a clinical practice guideline from the American College of Physicians. Ann Intern Med, 2005, 142, 525-531). The main causes of obesity are increased calorie intake accompanied with a lack of physical activity and genetic predisposition. Obesity leads to an increased risk of many diseases including, but not limited to, diabetes, heart disease, stroke, dementia, cancer, and osteoarthritis.

Metabolic syndrome is present when a group of risk factors are found in a mammal (Grundy, S. M.; Brewer, H. B. Jr.; et al., Circulation, 2004, 109, 433-438). Abdominal obesity, dyslipidemia, high blood pressure and insulin resistance predominate in this disease. Similar to obesity, metabolic syndrome results from increased calorie intake, physical inactivity, and aging. Of major concern is that this condition can lead to coronary artery disease and type 2 diabetes.

Clinically there are a number of treatments currently being used to lower blood glucose in type 2 diabetic patients. Metformin (De Fronzo, R. A.; Goodman, A. M., N. Engl. J. Med., 1995, 333, 541-549) and the PPAR agonists (Wilson, T. M., et al., J. Med. Chem., 1996, 39, 665-668) partially ameliorate insulin resistance by improving glucose utilization in cells. Treatment with sulfonylureas (Blickle, J. F., Diabetes Metab. 2006 32, 113-120) has been shown to promote insulin secretion by affecting the pancreatic KATP channel; however, the increase in insulin is not glucose dependent and such treatment can lead to hypoglycemia. The recently approved DPP4 inhibitors and GLP-1 mimetics promote insulin secretion by the beta cell through an incretin mechanism, and administration of these agents causes insulin release in a glucose dependent manner (Vahl, T. P., D'Alessio, D. A., Expert Opinion on Invest. Drugs, 2004, 13, 177-188). However, even with these newer treatments, it is difficult to achieve precise control of blood glucose levels in type 2 diabetic patients in accordance with the guidelines recommended by the American Diabetes Association.

GPR¹¹⁹ is a Gs-coupled receptor that is predominately expressed in the pancreatic beta cells and in the enteroendocrine K and L cells of the GI tract. In the gut, this receptor is activated by endogenous lipid-derived ligands such as oleoylethanolamide (Lauffer, L. M., et al., Diabetes, 2009, 58, 1058-1066). Upon activation of GPR¹¹⁹ by an agonist, the enteroendocrine cells release the gut hormones glucagon like peptide 1 (GLP-1), glucose-dependent insulinotropic peptide (GIP), and peptide YY (PYY) among others. GLP-1 and GIP have multiple mechanisms of action that are important for controlling blood glucose levels (Parker, H. E., et al., Diabetologia, 2009, 52, 289-298). One action of these hormones is to bind to GPCRs on the surface of beta cells leading to a rise in intracellular cAMP levels. This rise results in a glucose dependent release of insulin by the pancreas (Drucker, D. J. J. Clin. Investigation, 2007, 117, 24-32; Winzell, M. S., Pharmacol. and Therap. 2007, 116, 437-448). In addition, GLP-1 and GIP have been shown to increase beta cell proliferation and decrease the rate of apoptosis in vivo in animal models of diabetes and in vitro with human beta cells (Farilla, L.; et al., Endocrinology, 2002, 143, 4397-4408; Farilla, L.; et al., Endocrinology, 2003, 144 5149-5158; and Hughes, T. E., Current Opin. Chem. Biol., 2009, 13, 1-6). Current GLP-1 mechanism based therapies, such as sitagliptin and exenatide, are clinically validated to improve glucose control in type 2 diabetic patients.

GPR¹¹⁹ receptors are also expressed directly on the pancreatic beta cells. A GPR¹¹⁹ agonist can bind to the pancreatic GPR¹¹⁹ receptor and cause a rise in cellular cAMP levels consistent with a Gs-coupled GPCR signaling mechanism. The increased cAMP then leads to a release of insulin in a glucose dependent manner. The ability of GPR¹¹⁹ agonists to enhance glucose-dependent insulin release by direct action on the pancreas has been demonstrated in vitro and in vivo (Chu Z., et al., Endocrinology 2007, 148:2601-2609). This dual mechanism of action of the release of incretin hormones in the gut and binding directly to receptors on the pancreas may offer an advantage for GPR¹¹⁹ agonists over current therapies for treating diabetes.

GPR¹¹⁹ agonists, by increasing the release of PYY, may also be of benefit in treating many of comorbidities associated with diabetes and to treat these diseases in the absence of diabetes. Administration of PYY₃₋₃₆ has been reported to reduce food intake in animals (Batterham, R. L., et al., Nature, 2002, 418, 650-654), increase satiety and decrease food intake in humans (Batterham, R. L., et al., Nature, 2002, 418, 650-654), increase resting body metabolism (Sloth B., et al., Am. J. Physiol. Endocrinol. Metab., 2007, 292, E1062-1068 and Guo, Y., et al., Obesity, 2006, 14, 1562-1570), increase fat oxidation (Adams, S. H., et al., J. Nutr., 2006, 136, 195-201 and van den Hoek, A. M., et al., Diabetes, 2004, 53, 1949-1952), increase thyroid hormone activity, and increase adiponectin levels. PYY release caused by GPR¹¹⁹ agonists can therefore be beneficial in treating the metabolic syndrome and obesity.

Several classes of small molecule GPR¹¹⁹ agonists are known (Fyfe, M. T. E. et al., Expert Opin. Drug. Discov., 2008, 3(4), 403-413; Jones, R. M., et al., Expert Opin. Ther. Patents, 2009, 19(10), 1339-1359).

There remains, however, a need for compounds and methods for the treatment or prevention of diabetes, dyslipidemia, diabetic complications, and obesity.

### SUMMARY OF THE INVENTION

It has now been found that novel piperidinyl-substituted ureas are modulators of GPR¹¹⁹ and may be useful for treating type 2 diabetes, diabetic complications, metabolic syndrome, obesity, dyslipidemia, and related conditions.

Accordingly, in one aspect of the present invention there is provided compounds having the general Formula I

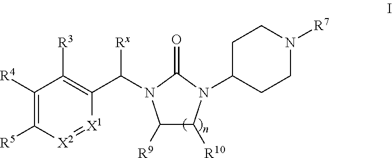

and pharmaceutically acceptable salts thereof, wherein X¹, X², R³, R⁴, R⁵, R^{x}, R⁷, R⁹, R¹⁰ and n are as defined herein.

In another aspect of the invention, there are provided pharmaceutical compositions comprising a compound of Formula I and a pharmaceutically acceptable carrier, diluent or excipient.

In another aspect of the invention, there is provided a method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I or a pharmaceutically acceptable salt thereof. In one embodiment, the disease is type 2 diabetes. In one embodiment, the method comprises administering a compound of Formula I in combination with one or more additional drugs. In one embodiment, the additional drug is a biguanide. In one embodiment, the additional drug is a DPP4 inhibitor.

In another aspect of the invention, there is provided the use of a compound of Formula I in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

In another aspect of the invention, there is provided compounds of Formula I or pharmaceutically acceptable salts thereof, for use in therapy.

In another aspect of the invention, there is provided compounds of Formula I or pharmaceutically acceptable salts thereof, for use in treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis, diabetic complications, metabolic syndrome (including hyperglycemia, impaired glucose tolerance, and insulin resistance), obesity, dyslipidemia, dyslipoproteinemia.

In another aspect of the invention, there is provided compounds of Formula I or pharmaceutically acceptable salts thereof, for use in treating type 2 diabetes.

Another aspect of the invention provides intermediates for preparing compounds of Formula I. In one embodiment, certain compounds of Formula I may be used as intermediates for the preparation of other compounds of Formula I.

Another aspect of the invention includes processes for preparing, methods of separation, and methods of purification of the compounds described herein.

### DETAILED DESCRIPTION OF THE INVENTION

One embodiment of this invention provides compounds of the general Formula I

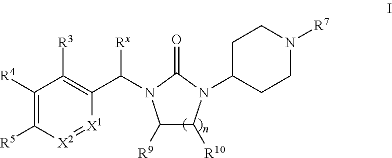

and pharmaceutically acceptable salts thereof, wherein:

X¹ is N or CR¹ and X² is N or CR², provided that only one of X¹ and X² is N;

R^{x} is H or (1-3C)alkyl;

R¹, R², R³ and R⁴ are independently selected from H, halogen, CF₃, (1-6C)alkyl, CN and (1-6C)alkoxy;

R⁵ is (1-3C alkyl)sulfonyl, (3-6C cycloalkyl)sulfonyl, (cyclopropylmethyl)sulfonyl, phenylsulfonyl, CN, R′R″NHC(═O)—, (1-5C)alkoxyC(═O)—, triazolyl, or tetrazolyl optionally substituted with (1-3C)alkyl;

R′ and R″ are independently H or (1-4C)alkyl optionally substituted with OH, or

R′ and R″ together with the atom to which they are attached form a 5-6 membered heterocyclic ring having a ring nitrogen atom and optionally having a second ring heteroatom selected from N and O, wherein said ring is optionally substituted with OH or NH₂;

R⁷ is selected from

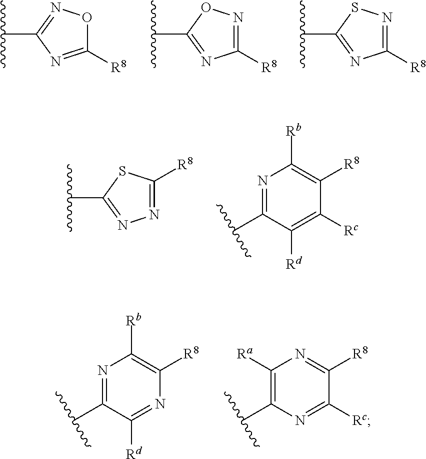

R^{a}, R^{b}, R^{c} and R^{d} are independently H or halogen;

R⁸ is selected from halogen, (1-6C)alkyl, fluoro(1-6C)alkyl, difluoro(1-6C)alkyl, and trifluoro(1-6C)alkyl;

R⁹ is hydrogen or (1-3C)alkyl;

R¹⁰ is hydrogen or (1-3C)alkyl; and

n is 1, 2 or 3, wherein when n is 2 or 3, only one of R¹⁰ can be methyl.

In one embodiment, Formula I includes compounds wherein:

X¹ is N or CR¹ and X² is N or CR², provided that only one of X¹ and X² is N;

R^{x} is H or (1-3C)alkyl;

R¹, R², R³ and R⁴ are independently selected from H, halogen, CF₃, (1-6C)alkyl, CN and (1-6C)alkoxy;

R⁵ is (1-3C alkyl)sulfonyl, (3-6C cycloalkyl)sulfonyl, (cyclopropylmethyl)sulfonyl, phenylsulfonyl, CN, R′R″NHC(═O)—, triazolyl, or tetrazolyl optionally substituted with (1-3C)alkyl;

R′ and R″ are independently H or (1-4C)alkyl;

R⁷ is selected from

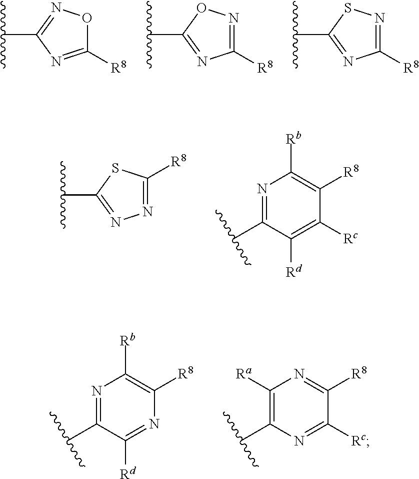

R^{a}, R^{b}, R^{c} and R^{d} are independently H or halogen;

R⁸ is selected from halogen, (1-6C)alkyl, fluoro(1-6C)alkyl, difluoro(1-6C)alkyl, and trifluoro(1-6C)alkyl;

R⁹ is hydrogen or (1-3C)alkyl;

R¹⁰ is hydrogen or (1-3C)alkyl; and

n is 1, 2 or 3, wherein when n is 2 or 3, only one of R¹⁰ can be methyl.

In one embodiment of Formula I, n is 1.

In one embodiment of Formula I, n is 2.

In one embodiment of Formula I, n is 3.

In one embodiment of Formula I, R^{x} is hydrogen.

In one embodiment of Formula I, R^{x} is methyl.

In one embodiment of Formula I, the residue:

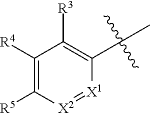

of Formula I, wherein the wavy line represents the point of attachment of the residue in Formula I, is selected from a residue wherein X¹ is CR¹ and X² is CR², such that the residue can be represented as:

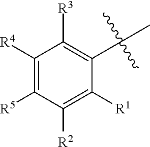

wherein R¹, R², R³, R⁴ and R⁵ are as defined for Formula I.

In one embodiment of Formula I, R¹, R², R³ and R⁴ are independently selected from H, (1-6C)alkyl, CF₃, CN and halogen. In one embodiment of Formula I, R¹, R², R³ and R⁴ are independently selected from H, (1-6C)alkyl, CF₃, and halogen. In one embodiment, R¹, R², R³ and R⁴ are independently selected from H and halogen.

In one embodiment, R¹ is H, F or Cl.

In one embodiment, R¹ is H.

In one embodiment, R¹ is F.

In one embodiment, R¹ is Cl.

In one embodiment, R² is H.

In one embodiment, R³ is H.

In one embodiment, R⁴ is H, Me, F, or Cl.

In one embodiment, R⁴ is H.

In one embodiment, R⁴ is Me.

In one embodiment, R⁴ is F.

In one embodiment, R⁴ is Cl.

In one embodiment, R¹ is F, and R², R³ and R⁴ are H.

In one embodiment, R¹ and R³ are F, and R² and R⁴ are H.

In one embodiment, R¹ and R⁴ are F and R² and R³ are H.

In one embodiment of Formula I, the residue:

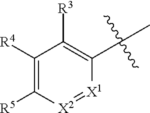

of Formula I, wherein the wavy line represents the point of attachment of the residue in Formula I, is selected from a residue wherein X¹ is N and X² is CR², such that the residue can be represented as:

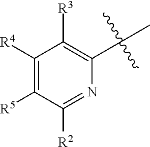

wherein R², R³, R⁴ and R⁵ are as defined for Formula I. In one embodiment, R², R³ and R⁴ are independently selected from H, F, Cl, CF₃, methyl, ethyl, propyl, isopropyl, methoxy, ethoxy, propoxy and isopropoxy. In one embodiment, R², R³ and R⁴ are independently selected from H, halogen, CF₃ and (1-6C)alkyl. In one embodiment, R², R³ and R⁴ are independently selected from H, halogen and (1-6C)alkyl. In one embodiment, R², R³ and R⁴ are independently selected from H, F, Cl and Me. In one embodiment, R², R³ and R⁴ are independently selected from H or Cl. In one embodiment, R², R³ and R⁴ are each H.

In one embodiment of Formula I, the residue:

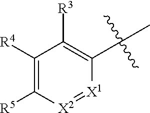

of Formula I, wherein the wavy line represents the point of attachment of the residue in Formula I, is selected from a residue wherein X¹ is CR¹ and X² is N, such that the residue can be represented as:

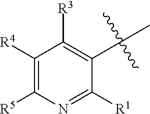

wherein R¹, R³, R⁴ and R⁵ are as defined for Formula I. In one embodiment, R¹, R³ and R⁴ are independently selected from H, F, Cl, CF₃, methyl, ethyl, propyl, isopropyl, methoxy, ethoxy, propoxy and isopropoxy. In one embodiment, R¹, R³ and R⁴ are independently selected from H, halogen, CF₃ and (1-6C)alkyl. In one embodiment, R¹, R³ and R⁴ are independently selected from H, halogen, and (1-6C)alkyl. In one embodiment, R¹, R³ and R⁴ are independently selected from H, F, Cl and Me. In one embodiment, each of R¹, R³ and R⁴ is H.

In one embodiment of Formula I, R⁵ is selected from (1-3C alkyl)sulfonyl, (3-6C cycloalkyl)sulfonyl, (cyclopropylmethyl)sulfonyl and phenylsulfonyl.

In one embodiment, R⁵ is (1-3C alkyl)sulfonyl. Examples include CH₃SO₂—, CH₃CH₂SO₂—, CH₃CH₂CH₂SO₂— and (CH₃)₂CHSO₂—. In one embodiment, R⁵ is CH₃SO₂—. In one embodiment, R⁵ is CH₃CH₂SO₂—. In one embodiment, R⁵ is (CH₃)₂CHSO₂—.

In one embodiment, R⁵ is (3-6C cycloalkyl)sulfonyl. An example is (cyclopropyl)SO₂—.

In one embodiment, R⁵ is (cyclopropylmethyl)sulfonyl which can be represented by the structure:

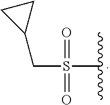 [SMILES: *S(=O)(=O)CC1CC1]

In one embodiment, R⁵ is phenylsulfonyl.

In one embodiment, R⁵ is CH₃SO₂—, CH₃CH₂SO₂—, CH₃CH₂CH₂SO₂—, (CH₃)₂CHSO₂—, (cyclopropyl)SO₂—, (cyclopropylmethyl)sulfonyl or phenylsulfonyl.

In one embodiment of Formula I, R⁵ is CN.

In one embodiment of Formula I, R⁵ is R′R″NHC(═O)—. In one embodiment, R⁵ is R′R″NHC(═O)— where R′ and R″ are independently H or (1-4C)alkyl optionally substituted with OH. In one embodiment, R′ and R″ are independently H, methyl, ethyl, or 2-hydroxyethyl. In one embodiment, R′ and R″ are independently (1-4C)alkyl. In one embodiment, R′ and R″ are independently H, methyl, or ethyl Particular examples of R⁵ are the structures:

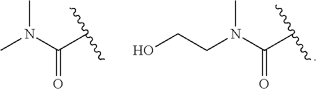

In one embodiment, R⁵ is R′R″NHC(═O)—, where R′ and R″ together with the atom to which they are attached form a 5-6 membered heterocyclic ring having a ring nitrogen atom and optionally having a second ring heteroatom selected from N and O, wherein said ring is optionally substituted with OH or NH₂. Particular examples of R⁵ include the structures:

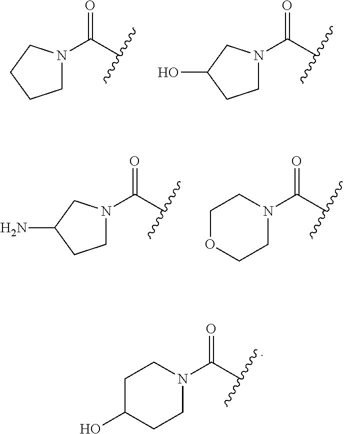

In one embodiment, R⁵ is (1-5C)alkoxyC(═O)—. In one embodiments, R⁵ is CH₃OC(═O)—.

In one embodiment of Formula I, R⁵ is triazolyl. A particular example of R⁵ is 1,2,4-triazol-1-yl.

In one embodiment of Formula I, R⁵ is tetrazolyl optionally substituted with (1-3C)alkyl. In one embodiment, R⁵ is tetrazolyl optionally substituted with methyl. Particular examples of R⁵ include groups having the structures:

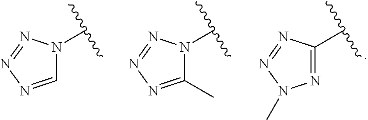

Examples of the group having the structure

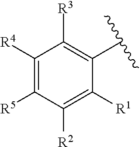

include the following structures:

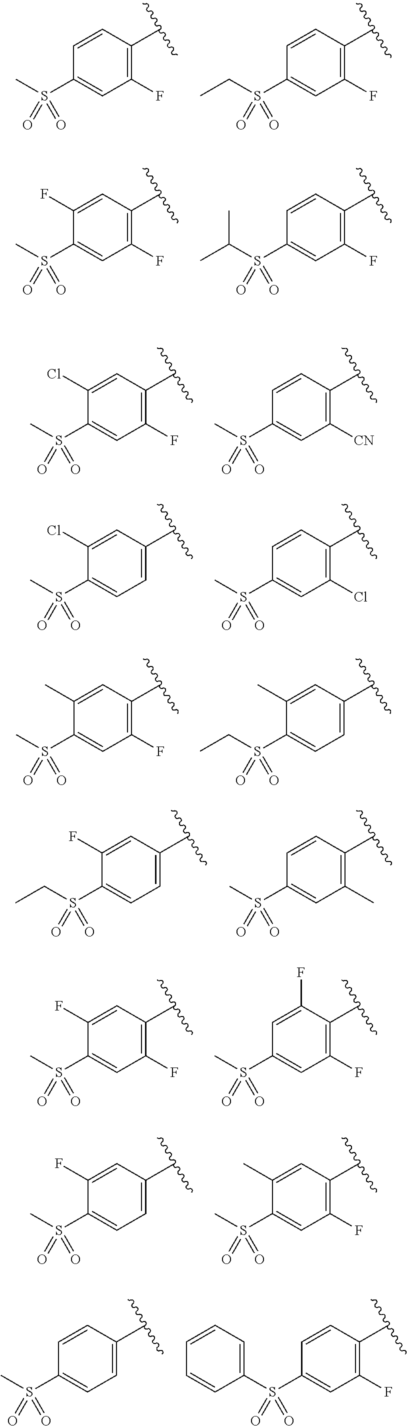

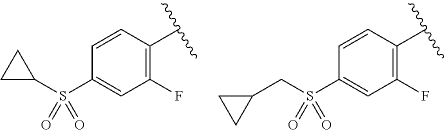

Particular examples the group having the structure

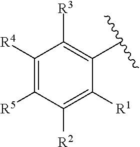

include the following structures:

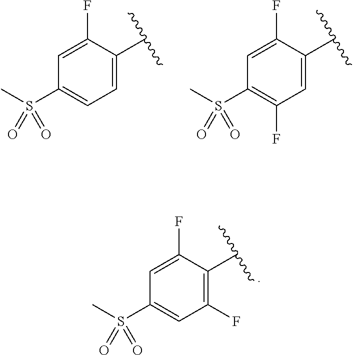 [SMILES: *c1ccc(S(C)(=O)=O)cc1F ; *c1cc(F)c(S(C)(=O)=O)cc1F ; *c1c(F)cc(S(C)(=O)=O)cc1F]

In one embodiment of Formula I, R⁷ is

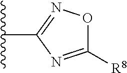

where R⁸ is as defined for Formula I.

In one embodiment of Formula I, R⁷ is

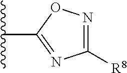

where R⁸ is as defined for Formula I.

In one embodiment of Formula I, R⁷ is

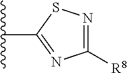

where R⁸ is as defined for Formula I.

In one embodiment of Formula I, R⁷ is

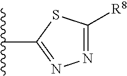

where R⁸ is as defined for Formula I.

In one embodiment of Formula I, R⁷ is

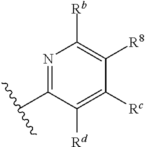

where R⁸ is as defined for Formula I.

In one embodiment of Formula I, R⁷ is

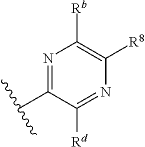

where R⁸ is as defined for Formula I.

In one embodiment of Formula I, R⁷ is

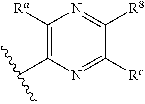

where R⁸ is as defined for Formula I.

In one embodiment of Formula I, R⁸ is (1-6C)alkyl. In one embodiment, R⁸ is methyl, ethyl, propyl, isopropyl, butyl, isobutyl or tert-butyl. In one embodiment, R⁸ is ethyl, isopropyl, sec-butyl or tert-butyl. In one embodiment, R⁸ is isopropyl.

In one embodiment of Formula I, R⁸ is fluoro(1-6C)alkyl. In one embodiment, R⁸ is 2-fluoropropyl.

In one embodiment of Formula I, R⁸ is difluoro(1-6C)alkyl. In one embodiment, R⁸ is difluoromethyl, 1,1-difluoroethyl or 1,1-difluoropropyl.

In one embodiment of Formula I, R⁸ is trifluoro(1-6C)alkyl. In one embodiment, R⁸ is trifluoromethyl or 1,1-dimethyl-2,2-difluoroethyl.

In one embodiment of Formula I, R⁸ is halogen. In one embodiment, R⁸ is Cl.

In one embodiment, R⁸ is selected from CF₃, Cl and isopropyl.

In one embodiment, R^{a}, R^{b}, R^{c} and R^{d} are hydrogen.

In one embodiment, R^{a}, R^{b}, R^{c} and R^{d} are halogen. In one embodiment, R^{a}, R^{b}, R^{c} and R^{d} are F.

In one embodiment, R^{a}, R^{b}, R^{c} and R^{d} are independently selected from H and F.

In one embodiment of Formula I, R⁷ is selected from

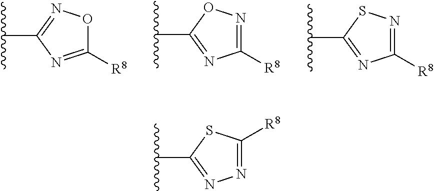

where R⁸ is as defined for Formula I. In one embodiment, R⁸ is selected from (1-6C)alkyl and trifluoro(1-6C)alkyl.

In one embodiment, R⁷ is selected from

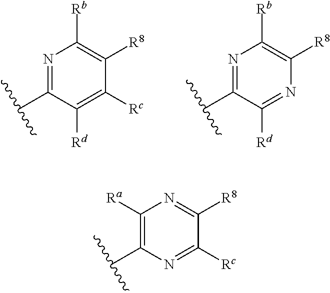

where R⁸, R^{a}, R^{b}, R^{c} and R^{d} are as defined for Formula I. In one embodiment, R⁸ is halogen or trifluoro(1-6C)alkyl and R^{a}, R^{b}, R^{c} and R^{d} are H or halogen

Particular examples of the group R⁷ include the structures:

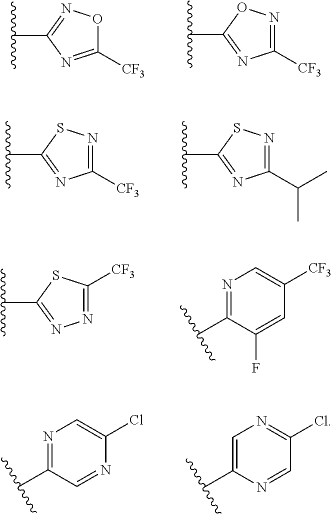

In one embodiment of Formula I, R⁹ is hydrogen.

In one embodiment of Formula I, R⁹ is (1-3C)alkyl. In one embodiment, R⁹ is methyl.

In one embodiment of Formula I, R¹⁰ is hydrogen.

In one embodiment of Formula I, R¹⁰ is (1-3C)alkyl. In one embodiment, R¹⁰ is methyl.

In one embodiment of Formula I, R⁹ is hydrogen and R¹⁰ is hydrogen.

In one embodiment of Formula I, R⁹ is hydrogen and R¹⁰ is methyl.

In one embodiment of Formula I, R⁹ is methyl and R¹⁰ is hydrogen.

Compounds of Formula I include compounds of Formula IA and pharmaceutically acceptable salts thereof, wherein:

R^{x} is H;

X¹ is CR¹ and X² is CR²;

R¹, R², R³ and R⁴ are independently selected from H and halogen;

R⁵ is (1-3C alkyl)sulfonyl, (3-6C cycloalkyl)sulfonyl or (cyclopropylmethyl)sulfonyl;

R⁷ is selected from

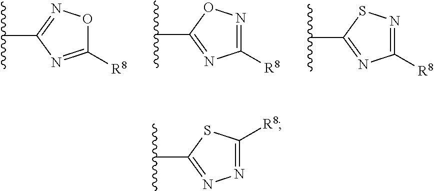

R⁸ is selected from (1-6C)alkyl, fluoro(1-6C)alkyl, difluoro(1-6C)alkyl, and trifluoro (1-6C)alkyl;

R⁹ is H;

R¹⁰ is H; and

n is 1, 2 or 3.

In one embodiment of Formula IA, R⁵ is (1-3C alkyl)sulfonyl.

Compounds of Formula I include compounds of Formula IB and pharmaceutically acceptable salts thereof, wherein:

R^{x} is H;

X¹ is CR¹ and X² is CR²;

R¹, R², R³ and R⁴ are independently selected from H and halogen;

R⁵ is (1-3C alkyl)sulfonyl, (3-6C cycloalkyl)sulfonyl or (cyclopropylmethyl)sulfonyl;

R⁷ is selected from

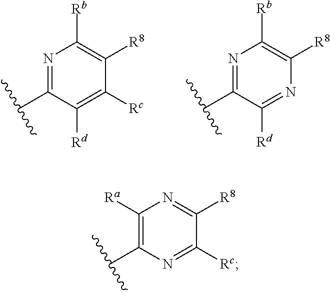

R^{a}, R^{b}, R^{c} and R^{d} are independently H or halogen;

R⁸ is selected from halogen, (1-6C)alkyl, fluoro(1-6C)alkyl, difluoro(1-6C)alkyl, and trifluoro(1-6C)alkyl;

R⁹ is H;

R¹⁰ is H; and

n is 1, 2 or 3.

In one embodiment of Formula IB, R⁵ is (1-3C alkyl)sulfonyl.

Particular compounds of the invention include:
1-(2,5-difluoro-4-(methylsulfonyl)benzyl)-3-(1-(5-(trifluoromethyl)-1,2,4-oxadiazol-3-yl)piperidin-4-yl)imidazolidin-2-one;
1-(2-fluoro-4-(methylsulfonyl)benzyl)-3-(1-(5-(trifluoromethyl)-1,2,4-oxadiazol-3-yl)piperidin-4-yl)imidazolidin-2-one;
1-(2,6-difluoro-4-(methylsulfonyl)benzyl)-3-(1-(5-(trifluoromethyl)-1,2,4-oxadiazol-3-yl)piperidin-4-yl)imidazolidin-2-one;
1-(2,5-difluoro-4-(methylsulfonyl)benzyl)-3-(1-(5-(trifluoromethyl)-1,2,4-oxadiazol-3-yl)piperidin-4-yl)tetrahydropyrimidin-2(1H)-one;
1-(2-fluoro-4-(methylsulfonyl)benzyl)-3-(1-(5-(trifluoromethyl)-1,2,4-oxadiazol-3-yl)piperidin-4-yl)tetrahydropyrimidin-2(1H)-one;
1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one;
Enantiomer 1 of 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one;
Enantiomer 2 of 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one;
1-(2,5-difluoro-4-(methylsulfonyl)benzyl)-3-(1-(3-(trifluoromethyl)-1,2,4-oxadiazol-5-yl)piperidin-4-yl)imidazolidin-2-one;
1-(2,5-difluoro-4-(methylsulfonyl)benzyl)-3-(1-(3-fluoro-5-(trifluoromethyl)pyridin-2-yl)piperidin-4-yl)imidazolidin-2-one;
1-(2,6-difluoro-4-(methylsulfonyl)benzyl)-3-(1-(3-fluoro-5-(trifluoromethyl)pyridin-2-yl)piperidin-4-yl)imidazolidin-2-one;
1-(1-(2,5-difluoro-4-(methylsulfonyl)phenyl)ethyl)-3-(1-(3-fluoro-5-(trifluoromethyl)pyridin-2-yl)piperidin-4-yl)imidazolidin-2-one;
1-(2-fluoro-4-(methylsulfonyl)benzyl)-3-(1-(3-fluoro-5-(trifluoromethyl)pyridin-2-yl)piperidin-4-yl)imidazolidin-2-one;
1-(2,5-difluoro-4-(methylsulfonyl)benzyl)-3-(1-(3-fluoro-5-(trifluoromethyl)pyridin-2-yl)piperidin-4-yl)tetrahydropyrimidin-2(1H)-one;
1-(2-fluoro-4-(methylsulfonyl)benzyl)-3-(1-(3-fluoro-5-(trifluoromethyl)pyridin-2-yl)piperidin-4-yl)tetrahydropyrimidin-2(1H)-one;
3-(2-fluoro-4-(methylsulfonyl)benzyl)-1-(1-(3-fluoro-5-(trifluoromethyl)pyridin-2-yl)piperidin-4-yl)-4-methylimidazolidin-2-one;
1-(2-fluoro-4-(methylsulfonyl)benzyl)-3-(1-(3-fluoro-5-(trifluoromethyl)pyridin-2-yl)piperidin-4-yl)-4-methylimidazolidin-2-one;
1-(2,5-difluoro-4-(methylsulfonyl)benzyl)-3-(1-(3-(trifluoromethyl)-1,2,4-thiadiazol-5-yl)piperidin-4-yl)imidazolidin-2-one;
1-(2-fluoro-4-(methylsulfonyl)benzyl)-3-(1-(3-(trifluoromethyl)-1,2,4-thiadiazol-5-yl)piperidin-4-yl)imidazolidin-2-one;
1-(2,6-difluoro-4-(methylsulfonyl)benzyl)-3-(1-(3-(trifluoromethyl)-1,2,4-thiadiazol-5-yl)piperidin-4-yl)imidazolidin-2-one;
1-(2,6-difluoro-4-(methylsulfonyl)benzyl)-3-(1-(5-(trifluoromethyl)-1,3,4-thiadiazol-2-yl)piperidin-4-yl)imidazolidin-2-one;
1-(2-fluoro-4-(methylsulfonyl)benzyl)-3-(1-(5-(trifluoromethyl)-1,3,4-thiadiazol-2-yl)piperidin-4-yl)imidazolidin-2-one;
1-(2,5-difluoro-4-(methylsulfonyl)benzyl)-3-(1-(5-(trifluoromethyl)-1,3,4-thiadiazol-2-yl)piperidin-4-yl)imidazolidin-2-one;
1-(1-(2,5-difluoro-4-(methylsulfonyl)phenyl)ethyl)-3-(1-(3-(trifluoromethyl)-1,2,4-thiadiazol-5-yl)piperidin-4-yl)imidazolidin-2-one;
1-(2,5-difluoro-4-(methylsulfonyl)benzyl)-3-(1-(3-(trifluoromethyl)-1,2,4-thiadiazol-5-yl)piperidin-4-yl)tetrahydropyrimidin-2(1H)-one;
1-(2-fluoro-4-(methylsulfonyl)benzyl)-3-(1-(3-(trifluoromethyl)-1,2,4-thiadiazol-5-yl)piperidin-4-yl)tetrahydropyrimidin-2(1H)-one;
3-(2-fluoro-4-(methylsulfonyl)benzyl)-4-methyl-1-(1-(3-(trifluoromethyl)-1,2,4-thiadiazol-5-yl)piperidin-4-yl)imidazolidin-2-one;
1-(2-fluoro-4-(methylsulfonyl)benzyl)-4-methyl-3-(1-(3-(trifluoromethyl)-1,2,4-thiadiazol-5-yl)piperidin-4-yl)imidazolidin-2-one;
1-(1-(5-chloropyrazin-2-yl)piperidin-4-yl)-3-(2,5-difluoro-4-(methylsulfonyl)benzyl)imidazolidin-2-one;
1-(1-(5-chloropyrazin-2-yl)piperidin-4-yl)-3-(2-fluoro-4-(methylsulfonyl)benzyl)imidazolidin-2-one;
1-(1-(5-chloropyrazin-2-yl)piperidin-4-yl)-3-(2,6-difluoro-4-(methylsulfonyl)benzyl)imidazolidin-2-one;
1-(2-fluoro-4-(methylsulfonyl)benzyl)-3-(1-(3-isopropyl-1,2,4-thiadiazol-5-yl)piperidin-4-yl)imidazolidin-2-one;
1-(2-fluoro-4-(methylsulfonyl)benzyl)-3-(1-(5-(trifluoromethyl)-1,3,4-thiadiazol-2-yl)piperidin-4-yl)tetrahydropyrimidin-2(1H)-one;
1-(2,6-difluoro-4-(methylsulfonyl)benzyl)-3-(1-(3-fluoro-5-(trifluoromethyl)pyridin-2-yl)piperidin-4-yl)tetrahydropyrimidin-2(1H)-one;
1-(2,6-difluoro-4-(methylsulfonyl)benzyl)-3-(1-(3-(trifluoromethyl)-1,2,4-thiadiazol-5-yl)piperidin-4-yl)tetrahydropyrimidin-2(1H)-one;
1-(1-(5-chloropyrazin-2-yl)piperidin-4-yl)-3-(2,6-difluoro-4-(methylsulfonyl)benzyl)tetrahydropyrimidin-2(1H)-one;
methyl 4-((3-(1-(3-fluoro-5-(trifluoromethyl)pyridin-2-yl)piperidin-4-yl)-2-oxoimidazolidin-1-yl)methyl)benzoate;
1-(1-(3-fluoro-5-(trifluoromethyl)pyridin-2-yl)piperidin-4-yl)-3-(4-(pyrrolidine-1-carbonyl)benzyl)imidazolidin-2-one;
1-(1-(3-fluoro-5-(trifluoromethyl)pyridin-2-yl)piperidin-4-yl)-3-(4-(4-hydroxypiperidine-1-carbonyl)benzyl)imidazolidin-2-one;
1-(1-(3-fluoro-5-(trifluoromethyl)pyridin-2-yl)piperidin-4-yl)-3-(4-(morpholine-4-carbonyl)benzyl)imidazolidin-2-one;
4-((3-(1-(3-fluoro-5-(trifluoromethyl)pyridin-2-yl)piperidin-4-yl)-2-oxoimidazolidin-1-yl)methyl)-N-methylbenzamide;
4-((3-(1-(3-fluoro-5-(trifluoromethyl)pyridin-2-yl)piperidin-4-yl)-2-oxoimidazolidin-1-yl)methyl)-N-isopropylbenzamide;
(S)-1-(1-(3-fluoro-5-(trifluoromethyl)pyridin-2-yl)piperidin-4-yl)-3-(4-(3-hydroxypyrrolidine-1-carbonyl)benzyl)imidazolidin-2-one;
(R)-1-(1-(3-fluoro-5-(trifluoromethyl)pyridin-2-yl)piperidin-4-yl)-3-(4-(3-hydroxypyrrolidine-1-carbonyl)benzyl)imidazolidin-2-one;
4-((3-(1-(3-fluoro-5-(trifluoromethyl)pyridin-2-yl)piperidin-4-yl)-2-oxoimidazolidin-1-yl)methyl)-N-(2-hydroxyethyl)-N-methylbenzamide;
(S)-1-(4-(3-aminopyrrolidine-1-carbonyl)benzyl)-3-(1-(3-fluoro-5-(trifluoromethyl)pyridin-2-yl)piperidin-4-yl)imidazolidin-2-one;
(R)-1-(4-(3-aminopyrrolidine-1-carbonyl)benzyl)-3-(1-(3-fluoro-5-(trifluoromethyl)pyridin-2-yl)piperidin-4-yl)imidazolidin-2-one;
3-fluoro-4-((3-(1-(3-fluoro-5-(trifluoromethyl)pyridin-2-yl)piperidin-4-yl)-2-oxoimidazolidin-1-yl)methyl)-N-(2-hydroxyethyl)-N-methylbenzamide;
1-(4-(1H-1,2,4-triazol-1-yl)benzyl)-3-(1-(3-fluoro-5-(trifluoromethyl)pyridin-2-yl)piperidin-4-yl)imidazolidin-2-one;
1-(1-(3-fluoro-5-(trifluoromethyl)pyridin-2-yl)piperidin-4-yl)-3-((6-(methylsulfonyl)pyridin-3-yl)methyl)imidazolidin-2-one;
1-(1-(3-fluoro-5-(trifluoromethyl)pyridin-2-yl)piperidin-4-yl)-3-((5-(methylsulfonyl)pyridin-2-yl)methyl)imidazolidin-2-one;
1-(4-(1H-tetrazol-1-yl)benzyl)-3-(1-(3-fluoro-5-(trifluoromethyl)pyridin-2-yl)piperidin-4-yl)imidazolidin-2-one;

and pharmaceutically acceptable salts thereof.

It will be appreciated that certain compounds according to the invention may contain one or more centers of asymmetry and may therefore be prepared and isolated as a mixture of isomers such as a racemic or diastereomeric mixture, or in an enantiomerically or diastereomerically pure form. It is intended that all stereoisomeric forms of the compounds of the invention, including but not limited to, diastereomers, enantiomers and atropisomers, as well as mixtures thereof such as racemic mixtures, form part of the present invention.

It may be advantageous to separate reaction products from one another and/or from starting materials. The desired products of each step or series of steps is separated and/or purified (hereinafter separated) to the desired degree of homogeneity by techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Enantiomers can be separated by converting the enantiomeric mixture into a diastereomeric mixture by reaction with an appropriate optically active compound (e.g., chiral auxiliary, such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column. Diastereomeric mixtures can be separated into their individual diastereomers on the basis of their physical chemical differences by methods well known to those skilled in the art, such as by chromatography and/or fractional crystallization.

A single stereoisomer, for example, an enantiomer, substantially free of its stereoisomer may be obtained by resolution of the racemic mixture using methods known in the art, such as (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., ed., Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E., and S. Wilen. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). An example of a chiral stationary phase is a CHIRALPAK ADH column. Enriched or purified enantiomers can be distinguished by methods used to distinguish other chiral molecules with asymmetric carbon atoms, such as optical rotation and circular dichroism.

It will further be appreciated that an enantiomer of a compound of the invention can be prepared by starting with the appropriate chiral starting material.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

In one embodiment, a compound of Formula I can be enriched in one enantiomer over the other by up to 80% enantiomeric excess. In one embodiment, a compound of Formula I can be enriched in one enantiomer over the other by up to 85% enantiomeric excess. In one embodiment, a compound of Formula I can be enriched in one enantiomer over the other by up to 90% enantiomeric excess. In one embodiment, a compound of Formula I can be enriched in one enantiomer over the other by up to 95% enantiomeric excess.

As used herein, the term “enantiomeric excess” means the absolute difference between the mole fraction of each enantiomer.

The term “(1-3C)alkyl” as used herein refer to saturated linear or branched-chain monovalent hydrocarbon radicals of one to three carbons, respectively.

The term “fluoro(1-6C)alkyl” as used herein refers to saturated linear or branched-chain monovalent radicals of one to six carbon atoms, wherein one of the hydrogen atoms is replaced by fluorine.

The term “difluoro(1-6C)alkyl” as used herein refers to saturated linear or branched-chain monovalent radicals of one to six carbon atoms, wherein two of the hydrogen atoms are replaced by fluorine.

The term “trifluoro(1-6C)alkyl” as used herein refers to saturated linear or branched-chain monovalent radicals of one to six carbon atoms wherein three of the hydrogen atoms are replaced by fluorine.

The term “(1-3C alkyl)sulfonyl” as used herein refers to a (1-3C alkyl)SO₂— group, wherein the radical is on the sulfur atom and the (1-3C alkyl) portion is as defined above.

The term “(3-6C cycloalkyl)sulfonyl” as used herein refers to a (3-6C cycloalkyl)SO₂— group, wherein the radical is on the sulfur atom. The term “(2-6C)dihydroxyalkyl” as used herein refers to saturated linear or branched-chain monovalent hydrocarbon radicals of two to six carbon atoms, respectively, wherein two of the hydrogen atoms are replaced with a OH group, provided that two OH groups are not on the same carbon.

The term “halogen” includes fluoro, chloro, bromo and iodo.

It will also be appreciated that certain compounds of Formula I may be used as intermediates for the preparation of further compounds of Formula I.

The compounds of Formula I include salts thereof. In certain embodiments, the salts are pharmaceutically acceptable salts. In addition, the compounds of Formula I include other salts of such compounds which are not necessarily pharmaceutically acceptable salts, and which may be useful as intermediates for preparing and/or purifying compounds of Formula I and/or for separating enantiomers of compounds of Formula I.

The term “pharmaceutically acceptable” indicates that the substance or composition is compatible chemically and/or toxicologically, with the other ingredients comprising a formulation, and/or the mammal being treated therewith.

It will further be appreciated that the compounds of Formula I and their salts may be isolated in the form of solvates, and accordingly that any such solvate is included within the scope of the present invention.

Compounds of the invention may also contain unnatural proportions of atomic isotopes at one or more of the atoms that constitute such compounds. That is, an atom, in particular when mentioned in relation to a compound according to Formula I, comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof. The compounds according to the invention therefore also comprise compounds with one or more isotopes of one or more atom, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds of the present invention, whether radioactive or not, are intended to be encompassed within the scope of the present invention.

The present invention further provides a process for the preparation of a compound of Formula I or a salt thereof as defined herein which comprises:

(a) reacting a corresponding compound of Formula II

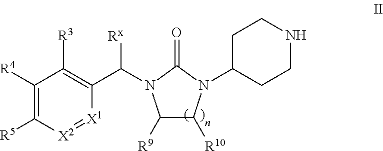

where X¹, X², R³, R⁴, R⁵, R^{x}, R⁹, R¹⁰ and n are as defined for Formula I, with a compound having the formula L¹-R⁷ where L¹ is a leaving atom and R⁷ is as defined for Formula I, in the presence of a base; or

(b) for a compound of Formula I where R⁷ is

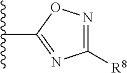

where R⁸ is as defined for Formula I, reacting a compound of Formula III

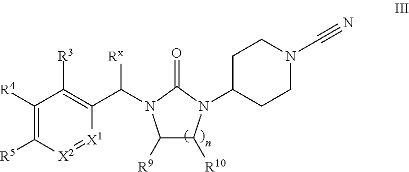

where X¹, X², R³, R⁴, R⁵, R^{x}, R⁹, R¹⁰ and n are as defined for Formula I, with a corresponding compound having the formula

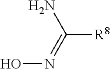

in the presence of a Lewis acid; or

(c) for a compound of Formula I where R⁷ is

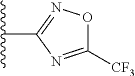 [SMILES: *c1noc(C(F)(F)F)n1]

reacting a corresponding compound of Formula III

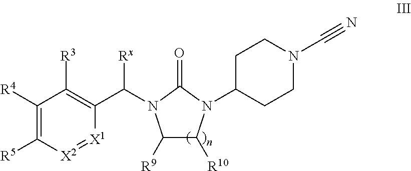

where X¹, X², R³, R⁴, R⁵, R^{x}, R⁹, R¹⁰ and n are as defined for Formula I, with hydroxylamine followed by treatment with 2,2,2-trifluoroacetic anhydride; or

(d) for a compound of Formula I wherein R⁵ is CN, reacting a corresponding compound having the formula IV

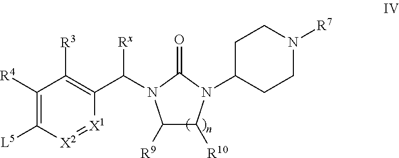

where L⁵ is a leaving group or atom, and X¹, X², R³, R⁴, R⁵, R^{x}, R⁹, R¹⁰ and n are as defined for Formula I, in the presence of a metal catalyst CuCN; or

(e) for a compound of Formula I wherein R⁵ is R′R′NHC(═O)—, and R′ and R″ together with the atom to which they are attached form a 5-6 membered heterocyclic ring having a ring nitrogen atom and optionally having a second ring heteroatom selected from N and O, wherein said ring is optionally substituted with OH or NH₂, reacting a corresponding compound having the formula V

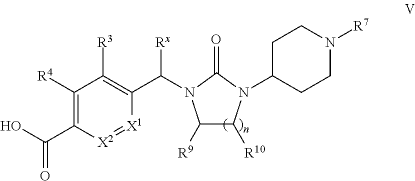

where X¹, X², R³, R⁴, R⁵, R^{x}, R⁹, R¹⁰ and n are as defined for Formula I, with a reagent having the formula

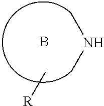

where ring B is a 5-6 membered heterocyclic ring having a ring nitrogen atom and optionally having a second ring heteroatom selected from N and O, wherein said ring is optionally substituted with OP¹ or NHP² where R¹ is hydrogen or a hydroxyl protecting group, and P² is hydrogen or an amino protecting group, in the presence of a coupling reagent; and

optionally removing any protecting groups and optionally preparing a salt thereof.

Referring to method (a), the leaving group may be a halogen, such as fluoro or chloro. Suitable bases include tertiary amine bases such as diisopropylethylamine (DIEA) and triethylamine. Convenient solvents include aprotic solvents such as DMF or ethers (for example tetrahydrofuran or p-dioxane). The reaction is conveniently performed at ambient temperature.

Referring to method (b), suitable Lewis acids include metal halides such as zinc chloride, aluminum chloride, or tin (IV) chloride. Suitable solvents include aprotic solvents such as ethers (for example tetrahydrofuran or p-dioxane). The reaction is conveniently performed at elevated temperatures, for example, between 50 and 150° C., for example 100° C.

Referring to method (c), suitable solvents include aprotic solvents such as ethers (for example tetrahydrofuran or dioxane). The reaction is conveniently performed at elevated temperatures, for example at 60° C.

Referring to method (d), L⁵ may be a leaving atom such as a halogen. Alternatively, L⁵ may be a leaving group such as an alkylsulfonyl or arylsulfonyl group, for example, a triflate group, an arylsulfonyloxy group or an alkylsulfonyloxy group, such as a mesylate or a tosylate group, NO₂, or a diazonium group. Suitable solvents include NMP. The reaction can be conveniently performed at elevated temperatures, for example temperatures ranging from 100-180° C., for example 160° C.

Referring to method (e), examples of suitable coupling reagents include (2-(7-aza-1H-benzotriazole-1-yl)-1,1,3,3-tetramethyluronium hexafluorophosphate) (HATU), HBTU, TBTU, DCC (N,N′-dicyclohexylcarbodiimide), DIEC (1-(3-dimethylaminopropyl)-3-ethylcarboiimide) and any other amide coupling reagents well known to persons skilled in the art.

Compounds of Formula III can be prepared by reacting a compound of Formula II with cyanic bromide.

In one embodiment, compounds of formulas II where R⁵ is (1-3C alkyl)sulfonyl, (3-6C cycloalkyl)sulfonyl, (cyclopropylmethyl)sulfonyl or phenylsulfonyl can be prepared as shown in general Scheme 1.

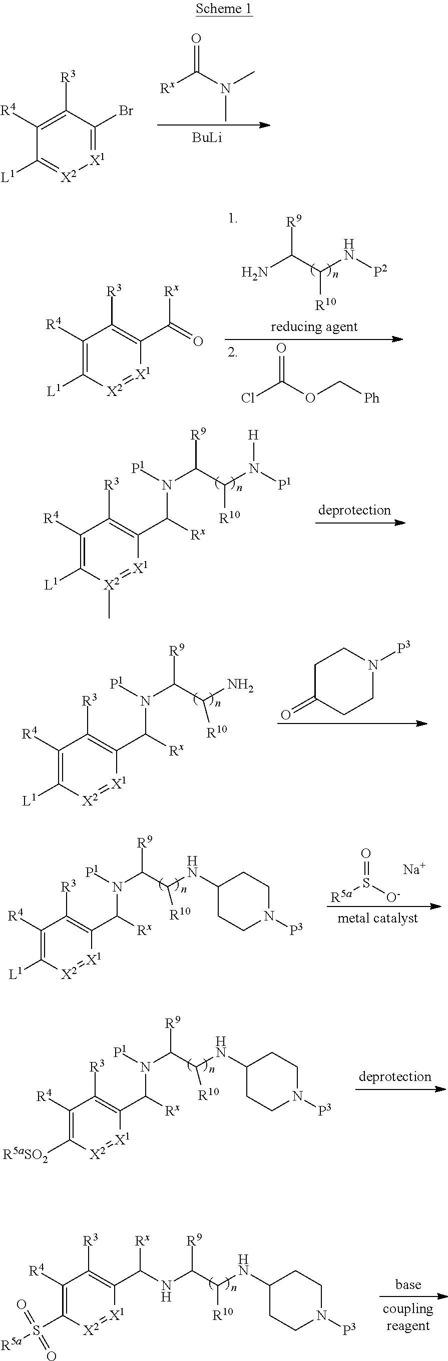

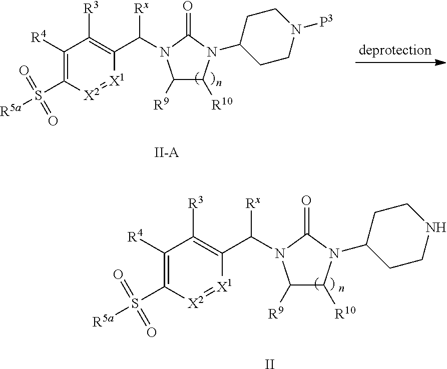

In Scheme 1, P¹, P² and P³ are amine protecting groups, R^{5′} is (1-3C)alkyl, (3-6C)cycloalkyl, cyclopropylmethyl or phenyl; and X¹, X², R³, R⁴, R^{x}, R⁷, R⁹, R¹⁰ and n are as defined for Formula I. In one embodiment, P³ is other than benzyl.

Amine groups in compounds described in any of the above methods may be protected with any convenient amine protecting group, for example as described in Greene & Wuts, eds., “Protecting Groups in organic Synthesis”, 2^{nd} ed. New York; John Wiley & Sons, Inc., 1991. Examples of amine protecting groups include acyl and alkoxycarbonyl groups, such as t-butoxycarbonyl (BOC), and [2-(trimethylsilyl)ethoxy]methyl (SEM). Additional examples of amine protecting groups include benzyl groups (—CH₂Ph) which may be unsubstituted or substituted. Likewise, carboxyl groups may be protected with any convenient carboxyl protecting group, for example as described in Greene & Wuts, eds., “Protecting Groups in organic Synthesis”, 2^{nd} ed. New York; John Wiley & Sons, Inc., 1991. Examples of carboxyl protecting groups include (1-6C)alkyl groups, such as methyl, ethyl and t-butyl. Alcohol groups may be protected with any convenient alcohol protecting group, for example as described in Greene & Wuts, eds., “Protecting Groups in organic Synthesis”, 2^{nd} ed. New York; John Wiley & Sons, Inc., 1991. Examples of alcohol (hydroxyl) protecting groups include benzyl, trityl, silyl ethers, and the like.

Compounds of the Formula II,

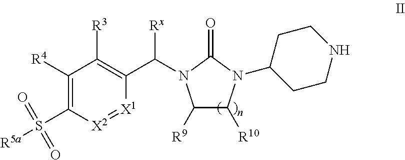

where X¹, X², R³, R⁴, R^{x}, R⁷, R⁹, R¹⁰ and n are as defined for Formula I; and R^{5a} is (1-3C)alkyl, (3-6C)cycloalkyl, cyclopropylmethyl or phenyl, are also believed to be novel and are provided as further aspects of the invention, with the proviso that Formula II does not include 1-[4-methylsulfphonyl)benzyl]-3-piperidin-4-ylimidazolidin-2-one,

Compounds of the Formula II-A

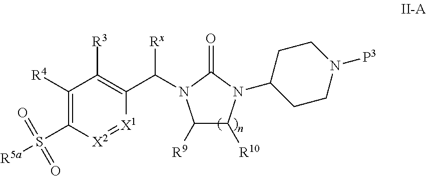

where X¹, X², R³, R⁴, R^{x}, R⁷, R⁹, R¹⁰ and n are as defined for Formula I; R^{5a} is (1-3C)alkyl, (3-6C)cycloalkyl, cyclopropylmethyl or phenyl; and P³ is other than benzyl, are also believed to be novel and are provided as further aspects of the invention. In one embodiment of Formula II-A, P³ is a Boc protecting group.

Compounds of the Formula III

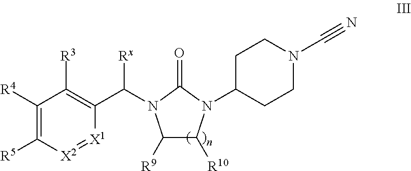

where X¹, X², R³, R⁴, R⁵, R^{x}, R⁷, R⁹, R¹⁰ and n are as defined for Formula I, are also believed to be novel and are provided as further aspects of the invention.

Compounds of the Formula IV where X¹, X², R³, R⁴, R^{x}, R⁷, R⁹, R¹⁰ and n are as defined for Formula I, are also believed to be novel and are provided as further aspects of the invention.

Compounds of Formula I are modulators of GPR119 and are useful for treating or preventing disease including, but not limited to, type 2 diabetes, diabetic complications, symptoms of diabetes, metabolic syndrome, obesity, dyslipidemia, and related conditions.

The ability of compounds of the invention to act as modulators of GPR119 may be demonstrated by the assay described in Example A.

The term “modulate” refers to the treating, prevention, suppression, enhancement or induction of a function or condition. For example, compounds can modulate Type 2 diabetes by increasing insulin in a human, thereby suppressing hyperglycemia.

The term “modulator” as used herein includes the terms agonist, antagonist, inverse agonist, and partial agonist.

The term “agonist” refers to a compound that binds to a receptor and triggers a response in a cell. An agonist mimics the effect of an endogenous ligand, a hormone for example, and produces a physiological response similar to that produced by the endogenous ligand.

The term “partial agonist” refers to a compound that binds to a receptor and triggers a partial response in a cell. A partial agonist produces only a partial physiological response of the endogenous ligand.

The term “antagonist” as used herein refers to is a type of receptor ligand or drug that does not provoke a biological response itself upon binding to a receptor, but blocks or dampens agonist-mediated responses.

The term “inverse agonist” as used herein refers to an agent that binds to the same receptor binding-site as an agonist for that receptor and reverses constitutive activity of the receptor.

Certain compounds of Formula I are agonists of GPR119.

Certain compounds of Formula I are inverse agonists of GPR119.

Certain compounds of Formula I are antagonists of GPR119.

In certain embodiments, compound of Formula I are useful for treating or preventing type 2 diabetes mellitus (also known as non-insulin dependent diabetes mellitus, or T2DM). Diabetes mellitus is a condition where the fasting plasma glucose level (glucose concentration in venous plasma) is greater than or equal to 126 mg/dL (tested on two occasions) and the 2-hour plasma glucose level of a 75 g oral glucose tolerance test (OGTT) is greater than or equal to 200 mg/dL. Additional classic symptoms include polydipsia, polyphagia and polyuria.

Accordingly, one aspect of the present invention provides methods for treating or preventing type 2 diabetes mellitus in a mammal, comprising administering to the mammal in need of such treatment a therapeutically effective amount of a compound of Formula I or a pharmaceutically acceptable salt thereof.

In certain embodiments, compound of Formula I are useful for treating or preventing diabetic complications. The term “diabetic complications” includes, but is not limited to, microvascular complications and macrovascular complications. Microvascular complications are those complications that generally result in small blood vessel damage. These complications include, for example, retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys). Macrovascular complications are those complications that generally result from large blood vessel damage. These complications include, e.g., cardiovascular disease and peripheral vascular disease. Cardiovascular disease is generally one of several forms, including, e.g., hypertension (also referred to as high blood pressure), coronary heart disease, stroke, and rheumatic heart disease. Peripheral vascular disease refers to diseases of any of the blood vessels outside of the heart. It is often a narrowing of the blood vessels that carry blood to leg and arm muscles.

Accordingly, one aspect of the present invention provides methods for treating or preventing diabetic complications in a mammal, comprising administering to the mammal in need of such treatment a therapeutically effective amount of a compound of Formula I or a pharmaceutically acceptable salt thereof. In one embodiment, the diabetic complication is retinopathy (also known as diabetic retinopathy).

In certain embodiments, compound of Formula I are useful for treating or preventing symptoms of diabetes. The term “symptom” of diabetes, includes, but is not limited to, polyuria, polydipsia, and polyphagia, as used herein, incorporating their common usage. For example, “polyuria” means the passage of a large volume of urine during a given period; “polydipsia” means chronic, excessive thirst; and “polyphagia” means excessive eating. Other symptoms of diabetes include, e.g., increased susceptibility to certain infections (especially fungal and staphylococcal infections), nausea, and ketoacidosis (enhanced production of ketone bodies in the blood).

Accordingly, one aspect of the present invention provides methods for treating or preventing symptoms of diabetes in a mammal, comprising administering to the mammal in need of such treatment a therapeutically effective amount of a compound of Formula I or a pharmaceutically acceptable salt thereof.

In certain embodiments, compound of Formula I are useful for treating or preventing metabolic syndrome in a mammal. The term “metabolic syndrome” refers to a cluster of metabolic abnormalities including abdominal obesity, insulin resistance, glucose intolerance, hypertension and dyslipidemia. These abnormalities are known to be associated with an increased risk of type 2 diabetes and cardiovascular disease. Compounds of Formula I are also useful for reducing the risks of adverse sequelae associated with metabolic syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

Accordingly, one aspect of the present invention provides methods of treating a metabolic syndrome in a mammal, comprising administering to the mammal in need of such treatment a therapeutically effective amount of a compound of Formula I or a pharmaceutically acceptable salt thereof. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

In certain embodiments, compound of Formula I are useful for treating or preventing obesity in a mammal. The term “obesity” refers to, according to the World Health Organization, a Body Mass Index (“BMI”) greater than 27.8 kg/m² for men and 27.3 kg/m² for women (BMI equals weight (kg)/height (m²)). Obesity is linked to a variety of medical conditions including diabetes and hyperlipidemia. Obesity is also a known risk factor for the development of Type 2 diabetes.

Accordingly, one aspect of the present invention provides methods of treating or preventing obesity in a mammal, comprising administering to the mammal in need of such treatment a therapeutically effective amount of a compound of Formula I or a pharmaceutically acceptable salt thereof.

Compounds of Formula I may also be useful for treating or preventing diseases and disorders such as, but not limited to, dyslipidemia and dyslipoproteinemia.

The term “dyslipidemia” refers to abnormal levels of lipoproteins in blood plasma including both depressed and/or elevated levels of lipoproteins (e.g., elevated levels of LDL and/or VLDL, and depressed levels of HDL).

The term “dyslipoproteinemia” refers to abnormal lipoproteins in the blood, including hyperlipidemia, hyperlipoproteinemia (excess of lipoproteins in the blood) including type I, II-a (hypercholesterolemia), II-b, III, IV (hypertriglyceridemia) and V (hypertriglyceridemia).

Accordingly, one aspect of the present invention provides methods of treating or preventing dyslipidemia in a mammal, comprising administering to the mammal in need of such treatment a therapeutically effective amount of a compound of Formula I or a pharmaceutically acceptable salt thereof.

Another aspect of the present invention provides methods of treating or preventing dyslipoproteinemia in a mammal, comprising administering to the mammal in need of such treatment a therapeutically effective amount of a compound of Formula I or a pharmaceutically acceptable salt thereof.

By elevating levels of active GLP-1 in vivo, the compounds are useful in treating neurological disorders such as Alzheimer's disease, multiple sclerosis, and schizophrenia.

Accordingly, one aspect of the invention provides methods of treating neurological disorders in a mammal, comprising administering to the mammal in need thereof a therapeutically effective amount of a compound of Formula I or a pharmaceutically acceptable salt thereof. In one embodiment, the neurological disorder is Alzheimer's disease.

Compounds of Formula I generally are useful for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

Accordingly, one aspect of the invention provides methods for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis, comprising administering to the mammal in need of such treatment a therapeutically effective amount of a compound of Formula I or a pharmaceutically acceptable salt thereof. In one embodiment, the disease is selected from type 2 diabetes.

According to another aspect, the invention provides methods for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia and dyslipoproteinemia.

Compounds of Formula I may also be useful for increasing satiety, reducing appetite, and reducing body weight in obese subjects and may therefore be useful in reducing the risk of co-morbidities associated with obesity such as hypertension, atherosclerosis, diabetes, and dyslipidemia.

Accordingly, the present invention provides methods of inducing satiety, reducing appetite, and reducing body weight in a mammal, comprising administering to the mammal in need thereof a therapeutically effective amount of a compound of Formula I or a pharmaceutically acceptable salt thereof.

In one aspect, the present invention provides methods of inducing satiety in a mammal, comprising administering to the mammal in need thereof a therapeutically effective amount of a compound of Formula I or a pharmaceutically acceptable salt thereof.

In one aspect, the present invention provides methods of decreasing food intake in a mammal, comprising administering to the mammal in need thereof a therapeutically effective amount of a compound of Formula I or a pharmaceutically acceptable salt thereof.

In one aspect, the present invention provides methods of controlling or decreasing weight gain of a mammal, comprising administering to the mammal in need thereof a therapeutically effective amount of a compound of Formula I or a pharmaceutically acceptable salt thereof.

Compounds of Formula I may be administered alone as a sole therapy or can be administered in addition with one or more other substances and/or treatments that work by the same or a different mechanism of action. These agents may be administered with one or more compounds of Formula I as part of the same or separate dosage forms, via the same or different routes of administration, and on the same or different administration schedules according to standard pharmaceutical practice known to one skilled in the art.

Accordingly, compounds of Formula I can be used in combination with a therapeutically effective amount of one or more additional drugs such as insulin preparations, agents for improving insulin resistance (for example PPAR gamma agonists), alpha-glucosidase inhibitors, biguanides (e.g., metformin), insulin secretagogues, dipeptidylpeptidase IV (DPP4) inhibitors (e.g., sitagliptin), beta-3 agonists, amylin agonists, phosphotyrosine phosphatase inhibitors, gluconeogenesis inhibitors, sodium-glucose cotransporter inhibitors, known therapeutic agents for diabetic complications, antihyperlipidemic agents, hypotensive agents, antiobesity agents, GLP-I, GIP-I, GLP-I analogs such as exendins, (for example exenatide (Byetta), exenatide-LAR, and liraglutide), and hydroxysterol dehydrogenase-1 (HSD-I) inhibitors. In one embodiment, a compound of Formula I is used in combination with a biguanide. In one embodiment, a compound of Formula I is used in combination with metformin. In one embodiment, a compound of Formula I is used in combination with metformin for the treatment of type 2 diabetes. In one embodiment, a compound as described in any one of the Examples is used in combination with metformin for the treatment of type 2 diabetes. In one embodiment, a compound of Formula I is used in combination with a DPP4 inhibitor. In one embodiment, a compound of Formula I is used in combination with sitagliptin. In one embodiment, a compound of Formula I is used in combination with sitagliptin for the treatment of type 2 diabetes. In one embodiment, a compound of any one of compounds of the Examples described below is used in combination with sitagliptin for the treatment of type 2 diabetes.

Accordingly, there is provided a method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I or a pharmaceutically acceptable salt thereof, in combination with in combination with a therapeutically effective amount of one or more additional drugs. In one embodiment, the combination is administered for the treatment of type 2 diabetes. In one embodiment, the additional drug is a biguanide. In one embodiment, the additional drug is metformin. In one embodiment, the additional drug is a DPP4 inhibitor. In one embodiment, the additional drug is sitagliptin.

As used herein, terms “treat” or “treatment” mean an alleviation, in whole or in part, of symptoms associated with a disorder or condition as described herein, or slowing, or halting of further progression or worsening of those symptoms. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be alleviated.

As used herein the terms “prevent” or “preventing” means the prevention of the onset, recurrence or spread, in whole or in part, of the disease or condition as described herein, or a symptom thereof.

The terms “effective amount” and “therapeutically effective amount” refer to an amount of compound that, when administered to a mammal in need of such treatment, is sufficient to (i) treat or prevent a particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound of Formula I that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the invention may be administered by any convenient route, e.g. into the gastrointestinal tract (e.g. rectally or orally), the nose, lungs, musculature or vasculature, or transdermally or dermally. Compounds may be administered in any convenient administrative form, for example tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, excipients and further active agents. If parenteral administration is desired, the compositions will be sterile and in a solution or suspension form suitable for injection or infusion. Such compositions form a further aspect of the invention.

In one embodiment, provided herein is a pharmaceutical combination comprising a therapeutically effective amount of: (a) at least one compound of Formula I; and (b) at least one agent selected from one or more additional drugs such as insulin preparations, agents for improving insulin resistance (for example PPAR gamma agonists), alpha-glucosidase inhibitors, biguanides (e.g., metformin), insulin secretagogues, dipeptidylpeptidase IV (DPP4) inhibitors (e.g., sitagliptin), beta-3 agonists, amylin agonists, phosphotyrosine phosphatase inhibitors, gluconeogenesis inhibitors, sodium-glucose cotransporter inhibitors, known therapeutic agents for diabetic complications, antihyperlipidemic agents, hypotensive agents, antiobesity agents, GLP-I, GIP-I, GLP-I analogs such as exendins, (for example exenatide (Byetta), exenatide-LAR, and liraglutide), and hydroxysterol dehydrogenase-1 (HSD-I) inhibitors, for treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, wherein (a) and (b) are in separate dosage forms or in the same dosage form. In one embodiment, the combination comprises (a) and (b) in an amount effective to treat type 2 diabetes, symptoms of diabetes, diabetic complications, or metabolic syndrome (including hyperglycemia, impaired glucose tolerance, and insulin resistance). In one embodiment, the combination comprises (a) and (b) in an amount effective to treat type 2 diabetes.

The term “pharmaceutical combination” as used herein means a product that results from the mixing or combining of more than one active ingredient and includes both fixed and non-fixed combinations of the active ingredients. The term “fixed combination” means that the active ingredients, e.g. (a) a compound of Formula I and (b) another agent, are both administered to a patient simultaneously in the form of a single entity or same dosage form. The term “non-fixed combination” means that the active ingredients, e.g. (a) a compound of Formula I and (b) another agent, are both administered to a patient as separate entities (separate dosage forms) either simultaneously, concurrently or sequentially with no specific time limits, wherein such administration provides therapeutically effective levels of the two compounds in the body of the patient. For a non-fixed combination, the individual combination partners of the combination may be administered separately at different times during the course of therapy or concurrently in divided or single combination forms.

The present invention further provides a pharmaceutical composition, which comprises a compound of Formula I or a pharmaceutically acceptable salt thereof, as defined hereinabove, and a pharmaceutically acceptable carrier, diluent or excipient.

An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

The present invention further provides a compound of Formula I or a pharmaceutically acceptable salt thereof, for use in therapy. In one embodiment, the present invention provides a compound of Formula I or a pharmaceutically acceptable salt thereof, for use in treating a disease or disorder selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis. In one embodiment, the present invention provides a compound of Formula I or a pharmaceutically acceptable salt thereof, for use in the treatment of type 2 diabetes, symptoms of diabetes, diabetic complications, metabolic syndrome (including hyperglycemia, impaired glucose tolerance, and insulin resistance), obesity, dyslipidemia, or dyslipoproteinemia. In one embodiment, the present invention provides a compound of Formula I or a pharmaceutically acceptable salt thereof, for use in the treatment of type 2 diabetes.

In one embodiment, the invention provides a compound of Formula I or a pharmaceutically acceptable salt thereof, for use in the treatment of a disease or disorder selected from type 2 diabetes, symptoms of diabetes, diabetic complications, metabolic syndrome (including hyperglycemia, impaired glucose tolerance, and insulin resistance), obesity, dyslipidemia, and dyslipoproteinemia.

In one embodiment, the invention provides a compound of Formula I or a pharmaceutically acceptable salt thereof, for use in the treatment of type 2 diabetes mellitus in a mammal.

In one embodiment, the invention provides a compound of Formula I or a pharmaceutically acceptable salt thereof, for use in the treatment of diabetic complications in a mammal.

In one embodiment, the invention provides a compound of Formula I or a pharmaceutically acceptable salt thereof, for use in the treatment of symptoms of diabetes in a mammal.

In one embodiment, the invention provides a compound of Formula I or a pharmaceutically acceptable salt thereof, for use in the treatment of metabolic syndrome in a mammal. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

In one embodiment, the invention provides a compound of Formula I or a pharmaceutically acceptable salt thereof, for use in the treatment of obesity in a mammal.

In one embodiment, the invention provides a compound of Formula I or a pharmaceutically acceptable salt thereof, for use in the treatment of dyslipidemia in a mammal.

In one embodiment, the invention provides a compound of Formula I or a pharmaceutically acceptable salt thereof, for use in the treatment of dyslipoproteinemia in a mammal.

In one embodiment, the invention provides a compound of Formula I or a pharmaceutically acceptable salt thereof, for use in the treatment of neurological disorders in a mammal. In one embodiment, the neurological disorder is Alzheimer's disease.

In one embodiment, the invention provides a compound of Formula I or a pharmaceutically acceptable salt thereof, for use in inducing satiety in a mammal.

In one embodiment, the invention provides a compound of Formula I or a pharmaceutically acceptable salt thereof, for use in decreasing food intake in a mammal.

In one embodiment, the invention provides a compound of Formula I or a pharmaceutically acceptable salt thereof, for use in controlling or decreasing weight gain in a mammal.

According to a further aspect, the present invention provides the use of a compound of Formula I or a pharmaceutically acceptable salt thereof, in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

According to a further aspect, the present invention provides the use of a compound of Formula I or a pharmaceutically acceptable salt thereof, in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, and dyslipoproteinemia,

According to a further aspect, the present invention provides the use of a compound of Formula I or a pharmaceutically acceptable salt thereof, in the treatment of type 2 diabetes mellitus in a mammal.

According to a further aspect, the present invention provides the use of a compound of Formula I or a pharmaceutically acceptable salt thereof, in the treatment of diabetic complications in a mammal.

According to a further aspect, the present invention provides the use of a compound of Formula I or a pharmaceutically acceptable salt thereof, in the treatment of symptoms of diabetes in a mammal.

According to a further aspect, the present invention provides the use of a compound of Formula I or a pharmaceutically acceptable salt thereof, in the treatment of metabolic syndrome in a mammal.

According to a further aspect, the present invention provides the use of a compound of Formula I or a pharmaceutically acceptable salt thereof, in the treatment of metabolic syndrome in a mammal. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

According to a further aspect, the present invention provides the use of a compound of Formula I or a pharmaceutically acceptable salt thereof, in the treatment of obesity in a mammal.

According to a further aspect, the present invention provides the use of a compound of Formula I or a pharmaceutically acceptable salt thereof, in the treatment of dyslipidemia in a mammal.

According to a further aspect, the present invention provides the use of a compound of Formula I or a pharmaceutically acceptable salt thereof, in the treatment of dyslipoproteinemia in a mammal.

According to a further aspect, the present invention provides the use of a compound of Formula I or a pharmaceutically acceptable salt thereof, in the treatment of neurological disorders in a mammal. In one embodiment, the neurological disorder is Alzheimer's disease.

According to a further aspect, the present invention provides the use of a compound of Formula I or a pharmaceutically acceptable salt thereof, in inducing satiety in a mammal.

According to a further aspect, the present invention provides the use of a compound of Formula I or a pharmaceutically acceptable salt thereof, in decreasing food intake in a mammal.

According to a further aspect, the present invention provides the use of a compound of Formula I or a pharmaceutically acceptable salt thereof, in controlling or decreasing weight gain in a mammal.

Another embodiment of the present invention provides the use of a compound of Formula I, or a pharmaceutically acceptable salt thereof, in the manufacture of a medicament for treating or preventing type 2 diabetes mellitus in a mammal.

Another embodiment of the present invention provides the use of a compound of Formula I, or a pharmaceutically acceptable salt thereof, in the manufacture of a medicament for treating or preventing diabetic complications.

Another embodiment of the present invention provides the use of a compound of Formula I, or a pharmaceutically acceptable salt thereof, in the manufacture of a medicament for treating or preventing symptoms of diabetes.

Another embodiment of the present invention provides the use of a compound of Formula I, or a pharmaceutically acceptable salt thereof, in the manufacture of a medicament for treating or preventing metabolic syndrome in a mammal.

Another embodiment of the present invention provides the use of a compound of Formula I, or a pharmaceutically acceptable salt thereof, in the manufacture of a medicament for treating or preventing obesity in a mammal.

Another embodiment of the present invention provides the use of a compound of Formula I, or a pharmaceutically acceptable salt thereof, in the manufacture of a medicament for treating or preventing dyslipidemia or dyslipoproteinemia.

Another embodiment of the present invention provides the use of a compound of Formula I, or a pharmaceutically acceptable salt thereof, in the manufacture of a medicament for treating neurological disorders in a mammal.

Another embodiment of the present invention provides the use of a compound of Formula I, or a pharmaceutically acceptable salt thereof, in the manufacture of a medicament for inducing satiety in a mammal.

Another embodiment of the present invention provides the use of a compound of Formula I, or a pharmaceutically acceptable salt thereof, in the manufacture of a medicament for decreasing food intake in a mammal.

Another embodiment of the present invention provides the use of a compound of Formula I, or a pharmaceutically acceptable salt thereof, in the manufacture of a medicament for controlling or decreasing weight gain of a mammal.

In one embodiment, the compound of Formula I is selected from any one of the compounds of Examples 1-67 or a pharmaceutically acceptable salt thereof. In one embodiment, the pharmaceutically acceptable salt is a trifluoroacetate and hydrochloride salts.

### EXAMPLES

The following examples illustrate the invention. In the examples described below, unless otherwise indicated all temperatures are set forth in degrees Celsius. Reagents were purchased from commercial suppliers such as Aldrich Chemical Company, Lancaster, Alfa, Aesar, TCI, Maybridge, or other suitable suppliers, and were used without further purification unless otherwise indicated. THF, DCM, toluene, DMF and dioxane were purchased from commercial vendors and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried or dried under a stream of dry nitrogen.

Column chromatography was done on a Biotage system (Manufacturer: Dyax Corporation) having a silica gel or C-18 reverse phase column, or on a silica SepPak cartridge (Waters), or using conventional flash column chromatography on silica gel, unless otherwise specified.

### Example A

### cAMP Production Assay

The assay utilized HEK-293 cells that stably express a modified version of the GPR119 receptor (94% identity to human receptor), under the control of a CMV promoter containing a tet-on element for tetracycline-inducible expression. GPR119 agonist-induced cyclic AMP (cAMP) production was measured in this cell line using the LANCE cAMP kit (Perkin Elmer, Waltham, Mass.). To generate a working stock of cells for the assay, cells were treated overnight with 1 μg/mL doxycycline at 37° C. in the presence of 5% CO₂ to induce receptor expression. Cells were then harvested by enzymatic dissociation with 0.05% trypsin, resuspended in freezing medium (DMEM growth medium with 10% each of fetal bovine serum and DMSO), aliquoted and frozen at −80° C. On the day of the assay, frozen cells were thawed, washed 1× in PBS and resuspended in Hank's buffered salt solution (HBSS) containing 5 mM HEPES, 0.1% BSA and Alexa Fluor 647-conjugated anti-cAMP antibody (diluted 1:100). The cell suspension was then transferred to a Proxiplate Plus white 384-well assay plate (Perkin-Elmer) at 2000 cells/well. Test compounds at final concentrations ranging from 0.2 nM to 10 μM were added to the assay plate, followed by a one-hour incubation at ambient temperature (volume=10 μL/well). DMSO concentration was held constant at 0.5%. After incubation with test compounds, 10 μL of a detergent buffer containing a biotinylated cAMP/Europium-conjugated streptavidin complex (Europium-labeled cAMP tracer) were added to each well on the assay plate, followed by a 2-hour incubation at ambient temperature. During this incubation cAMP released from lysed cells competes with the Europium-labeled cAMP tracer for binding to the Alexa Fluor 647-conjugated antibody. Agonist-induced cellular cAMP production resulted in increased competition with the Europium-labeled cAMP tracer, leading to a proportional decrease in the time-resolved fluorescence resonance energy transfer (TR-FRET) signal detected by the Perkin-Elmer Envision plate reader. Cellular cAMP levels were then determined by interpolation of raw signal data using a cAMP standard curve. Compounds were determined to have agonist activity if they stimulated a 1.5-fold or greater increase in cAMP relative to basal levels. Results for the compounds of Examples 1-32 are shown in Table A.

TABLE A

| | cAMP production in | |
|---|---|---|
| | HEK-293 cells | |
| Example No. | (nMol) | Fold over baseline |
| 1 | 3.00 | 14.4 |
| 2 | 3.64 | 13.2 |
| 3 | 3.76 | 24.3 |
| 4 | 3.0 | 16.4 |
| 5 | 2.05 | 5.9 |
| 6 | 2.84 | 14.7 |
| 7 | 3.90 | 7.6 |
| 8 | 5.62 | 9.7 |
| 9 | 3.05 | 20.3 |
| 10 | 2.83 | 12.8 |
| 11 | 3.41 | 22.7 |
| 12 | 2.73 | 16.78 |
| 13 | 4.06 | 4.9 |
| 14 | 2.34 | 12.2 |
| 15 | 1.79 | 5 |
| 16 | 2.64 | 13.1 |
| 17 | 2.34 | 11.5 |
| 18 | 2.77 | 12.2 |
| 19 | 3.78 | 13.6 |
| 20 | 2.24 | 12.3 |
| 21 | 3.37 | 28.21 |
| 22 | 3.42 | 31.88 |
| 23 | 3.12 | 19.7 |
| 24 | 2.19 | 11.4 |
| 25 | 1.78 | 7.9 |
| 26 | 1.49 | 2.78 |
| 27 | 2.11 | 9 |
| 28 | 2.41 | 11.3 |
| 29 | 2.98 | 19.63 |
| 30 | 4.58 | 17.2 |
| 31 | 3.35 | 23.3 |
| 32 | 5.35 | 20 |
| 33 | 2.36 | 8.15 |
| 34 | 2.72 | 4.3 |
| 35 | 2.36 | 2.86 |
| 36 | 3.54 | 6.36 |
| 37 | 2.14 | 3.31 |
| 38 | 5.46 | 11.6 |
| 39 | 3.30 | 6.21 |
| 40 | 4.73 | 9.7 |
| 41 | 4.25 | 8.46 |
| 42 | 3.23 | 5.81 |
| 43 | 4.45 | 9.33 |
| 44 | 3.99 | 8.07 |
| 45 | 4.17 | 7.6 |
| 46 | 1.70 | 1.83 |
| 47 | 2.17 | 4.22 |
| 48 | 3.72 | 29.4 |
| 49 | 4.58 | 9.67 |
| 50 | 3.75 | 7.98 |
| 51 | 7.56 | 11.81 |
| 52 | 3.96 | 32.9 |

### Intermediate 1

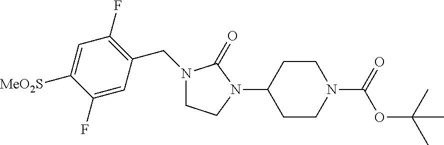 [SMILES: CC(C)(C)OC(=O)N1CCC(N2CCN(Cc3cc(F)c(S(C)(=O)=O)cc3F)C2=O)CC1]

### tert-butyl 4-(3-(2,5-difluoro-4-(methylsulfonyl)benzyl)-2-oxoimidazolidin-1-yl)piperidine-1-carboxylate

Step 1: To a solution of 1,4-dibromo-2,5-difluorobenzene (15 g, 55 mmol) in toluene (600 mL) cooled to −78° C. was added butyllithium (22 mL, 55 mmol) and the reaction was kept at −78° C. for 30 minutes. N,N-dimethylformamide (4.4 g, 61 mmol) was added and the reaction was stirred for 2 hours while warming to ambient temperature. To the reaction was added water (200 mL) and EtOAc (400 mL) and the organic layers were separated. The organic layer was washed with brine (200 mL), dried over MgSO₄, filtered and concentrated in vacuo. The crude material was chromatographed eluting with 5% EtOAc/Hexane to yield 4-bromo-2,5-difluorobenzaldehyde (6.7 g, 55.8%).

Step 2: To a solution of tert-butyl 2-aminoethylcarbamate (4.86 g, 30.3 mmol) in THF (500 mL) was added 4-bromo-2,5-difluorobenzaldehyde (6.7 g, 30.3 mmol) followed by acetic acid (1.93 mL, 33.3 mmol) and the reaction was stirred for 30 minutes. Sodium triacetoxyhydroborate (9.64 g, 45.5 mmol) was added and the reaction was stirred overnight at ambient temperature. To the reaction was added sodium hydrogen carbonate (121 mL, 121 mmol, a saturated aqueous solution) and the reaction was stirred for 30 minutes followed by addition of benzyl carbonochloridate (4.31 mL, 30.3 mmol). The reaction was stirred at ambient temperature for 4 hours. The reaction was partitioned between EtOAc (500 mL) and water (500 mL) and the organic layer was separated. The organic layer was washed with brine (100 mL), dried over MgSO₄, filtered and concentrated in vacuo. The crude material was chromatographed eluting with DCM to give benzyl 4-bromo-2,5-difluorobenzyl(2-((tert-butoxycarbonyl)amino)ethyl)carbamate (13 g, 26.0 mmol, 85.9% yield).

Step 3: To a solution of benzyl 4-bromo-2,5-difluorobenzyl(2-((tert-butoxycarbonyl)amino)ethyl)carbamate (13 g, 26 mmol) in DCM (200 mL) was added 2,2,2-trifluoroacetic acid (15 g, 130 mmol) and the reaction was stirred at ambient temperature for 1 hour. The organic layer was concentrated in vacuo to give benzyl 2-aminoethyl(4-bromo-2,5-difluorobenzyl)carbamate (10 g, 25 mmol, 96% yield, as the TFA salt).

Step 4: To a solution of benzyl 2-aminoethyl(4-bromo-2,5-difluorobenzyl)carbamate (as the TFA salt) (10 g, 25 mmol) in THF (100 mL) was added tert-butyl 4-oxopiperidine-1-carboxylate (5.5 g, 28 mmol) followed by acetic acid (1.8 g, 10 mmol), and the reaction was stirred at ambient temperature for 30 minutes. NaBH(OAc)₃ (11 g, 50 mmol) was added and the reaction was stirred overnight at ambient temperature. The reaction was quenched with a saturated aqueous solution of NaHCO₃ (200 mL) and then stirred for 30 minutes. The reaction was diluted with EtOAc (500 mL) and the organic layer was washed with 1N NaOH (100 mL) and basic brine (100 mL), dried over Na₂SO₄ and concentrated in vacuo. The crude material was chromatographed eluting with 2% MeOH/EtOAc to yield tert-butyl 4-(2-((benzyloxycarbonyl)(4-bromo-2,5-difluorobenzyl)amino)ethylamino)piperidine-1-carboxylate (15 g, 26 mmol, 103% yield).

Step 5: To a solution of tert-butyl 4-(2-((benzyloxycarbonyl)(4-bromo-2,5-difluorobenzyl)amino)ethylamino)piperidine-1-carboxylate (15 g, 26 mmol) in DMSO (70 mL) was added sodium methanesulfinate (3.9 g, 39 mmol) and cyclohexane-1,2-diamine (1.2 g, 10 mmol) and the reaction was degassed with nitrogen for 40 minutes. Cu(I) triflate benzene complex (1.3 g, 2.6 mmol) was added and the reaction was stirred overnight at 110° C. The reaction was diluted with EtOAc (500 mL). The organic layer was washed with saturated aqueous NaHCO₃ and basic brine, dried over Na₂SO₄, filtered and concentrated in vacuo. The crude material was chromatographed eluting with 5% MeOH/EtOAc to yield tert-butyl 4-(2-((benzyloxycarbonyl)(2,5-difluoro-4-(methylsulfonyl)benzyl)amino)ethylamino) piperidine-1-carboxylate (6.3 g, 11 mmol, 42% yield).

Step 6: To a solution of tert-butyl 4-(2-((benzyloxycarbonyl)(2,5-difluoro-4-(methylsulfonyl)benzyl)amino)ethylamino)piperidine-1-carboxylate (6.3 g, 11 mmol) in methanol (100 mL) was added 500 mg 10% Pd/C (Degussa type, 50% water) and the reaction degassed with nitrogen. The reaction was next degassed with hydrogen and held under an atmosphere of hydrogen for 2 hours. The reaction was degassed with nitrogen. The slurry was filtered through Celite® and the filtrate was concentrated in vacuo to give tert-butyl 4-(2-(2,5-difluoro-4-(methylsulfonyl)benzylamino)ethylamino)piperidine-1-carboxylate (4.2 g, 87%).

Step 7: To a solution of tert-butyl 4-(2-(2,5-difluoro-4-(methylsulfonyl)benzylamino)ethylamino)piperidine-1-carboxylate (4.2 g, 9.38 mmol) in THF (100 mL) was added N-ethyl-N-isopropylpropan-2-amine (3.43 mL, 18.8 mmol) and di(1H-imidazol-1-yl)methanone (3.04 g, 18.8 mmol), and the reaction was heated to 60° C. for 4 hours. The reaction was diluted with EtOAc (500 mL), washed with water (100 mL) and brine (100 mL), dried over MgSO₄, filtered and concentrated in vacuo. The crude material was chromatographed eluting with 1:1 EtOAc/Hexane to give tert-butyl 4-(3-(2,5-difluoro-4-(methylsulfonyl)benzyl)-2-oxoimidazolidin-1-yl)piperidine-1-carboxylate (3.3 g, 6.97 mmol, 74.3% yield). Mass spectrum 374.1 (M+H-Boc).

### Intermediate 2

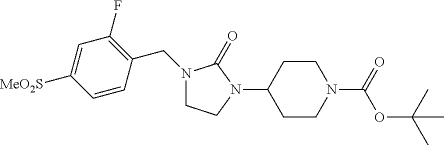 [SMILES: CC(C)(C)OC(=O)N1CCC(N2CCN(Cc3ccc(S(C)(=O)=O)cc3F)C2=O)CC1]

### tert-butyl 4-(3-(2-fluoro-4-(methylsulfonyl)benzyl)-2-oxoimidazolidin-1-yl)piperidine-1-carboxylate

Prepared according to the method described for Intermediate 1, omitting step 1 and substituting 4-bromo-2-fluorobenzaldehyde for 4-bromo-2,5-difluorobenzaldehyde in step 2. Mass spectrum 356.1 (M+H-Boc).

### Intermediate 3

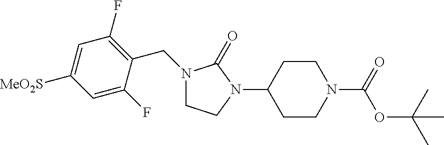 [SMILES: CC(C)(C)OC(=O)N1CCC(N2CCN(Cc3c(F)cc(S(C)(=O)=O)cc3F)C2=O)CC1]

### tert-butyl 4-(3-(2,6-difluoro-4-(methylsulfonyl)benzyl)-2-oxoimidazolidin-1-yl)piperidine-1-carboxylate

Prepared according to the method described for Intermediate 1, omitting step 1 and substituting 4-bromo-2,6-difluorobenzaldehyde for 4-bromo-2,5-difluorobenzaldehyde in Step 2. Mass spectrum 374.1 (M+H-Boc).

### Intermediate 4

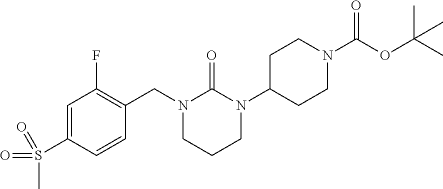 [SMILES: CC(C)(C)OC(=O)N1CCC(N2CCCN(Cc3ccc(S(C)(=O)=O)cc3F)C2=O)CC1]

### tert-butyl 4-(3-(2-fluoro-4-(methylsulfonyl)benzyl)-2-oxotetrahydropyrimidin-1(2H)-yl)piperidine-1-carboxylate

Step 1: To a solution of tert-butyl 3-aminopropylcarbamate (8.58 g, 49.3 mmol) in THF (125 mL) was added 4-bromo-2-fluorobenzaldehyde (10 g, 49.3 mmol) followed by acetic acid (3.13 mL, 54.2 mmol) and the reaction was stirred for 30 minutes. Sodium triacetoxyhydroborate (15.7 g, 73.9 mmol) was added and the reaction was stirred overnight at ambient temperature. To the reaction was added a saturated aqueous solution of sodium hydrogen carbonate (246 mL) and the reaction was stirred for 20 minutes. Benzyl carbonochloridate (7.00 mL, 49.3 mmol) was added and the reaction was stirred at ambient temperature for 2 hours. The reaction was partitioned between EtOAc (400 mL) and water (200 mL) and the organic layers were separated. The organic layer was washed with brine (100 mL), dried over MgSO₄, filtered and concentrated in vacuo to give benzyl 4-bromo-2-fluorobenzyl(3-((tert-butoxycarbonyl)amino)propyl)carbamate (24.5 g, 100%).

Step 2: To a solution of benzyl 4-bromo-2-fluorobenzyl(3-((tert-butoxycarbonyl)amino)propyl)carbamate (24 g, 48.4 mmol) in DCM (100 mL) was added 2,2,2-trifluoroacetic acid (27.6 g, 242 mmol) and the reaction was stirred at ambient temperature for 1 hour. The reaction was concentrated in vacuo. The organic layer was diluted with EtOAc (200 mL) and a saturated aqueous solution of Na₂CO₃ (100 mL) and the layers were separated. The organic layer was concentrated in vacuo to give benzyl 3-aminopropyl(4-bromo-2-fluorobenzyl)carbamate (19.1 g, 100%).

Step 3: To a solution of benzyl 3-aminopropyl(4-bromo-2-fluorobenzyl)carbamate (19.0 g, 43.3 mmol) in THF (250 mL) was added tert-butyl 4-oxopiperidine-1-carboxylate (9.48 g, 47.6 mmol) and acetic acid (3.12 g, 51.9 mmol) and the reaction was stirred for 30 minutes. NaBH(OAc)₃ (18.3 g, 86.5 mmol) was added and the reaction was stirred overnight at ambient temperature. To the reaction was added a saturated aqueous NaHCO₃ solution (500 mL), followed by EtOAc (500 mL) and the reaction was stirred for 30 minutes. The organics were separated, washed with brine (100 mL), dried over sodium sulfate and concentrated in vacuo to give tert-butyl 4-(3-((benzyloxycarbonyl)(4-bromo-2-fluorobenzyl)amino)propylamino)piperidine-1-carboxylate (26 g, 100%).

Step 4: To a solution of tert-butyl 4-(3-((benzyloxycarbonyl)(4-bromo-2-fluorobenzyl)amino)propylamino)piperidine-1-carboxylate (9.0 g, 16 mmol) in DMSO (100 mL) was added cyclohexane-1,2-diamine (0.71 g, 6.2 mmol) and sodium methanesulfinate (2.4 g, 23 mmol). The reaction was degassed with nitrogen for 1 hour, Cu(I) triflate benzene complex (0.78 g, 1.6 mmol) was added and the reaction was heated to 110° C. overnight. The reaction was diluted with EtOAc and washed with NaOH (1M, 2×100 mL) and basic brine (100 mL), dried over Na₂SO₄ filtered and concentrated in vacuo. The crude material was chromatographed eluting with 5% MeOH/EtOAc to give tert-butyl 4-(3-((benzyloxycarbonyl)(2-fluoro-4-(methylsulfonyl)benzyl)amino)propylamino)piperidine-1-carboxylate (5 g, 8.7 mmol, 56% yield).

Step 5: To a solution of tert-butyl 4-(3-((benzyloxycarbonyl)(2-fluoro-4-(methylsulfonyl)benzyl)amino)propylamino)piperidine-1-carboxylate (5 g, 8.7 mmol) in methanol (100 mL) was added 0.5 g of 10% Pd/C (Degussa type, 50% water) and the reaction was stirred under a balloon of hydrogen overnight. The reaction was degassed with N₂ followed by addition of another 300 mg of 10% Pd/C (degussa type, 50% water) and the reaction was stirred under a hydrogen balloon overnight. The reaction was degassed using N₂ and the reaction filtered through Celite®. The combined organic layers were concentrated in vacuo to give tert-butyl 4-(3-(2-fluoro-4-(methylsulfonyl)benzylamino)propylamino) piperidine-1-carboxylate (4.0 g, 9.0 mmol, 104% yield).

Step 6: To a solution of tert-butyl tert-butyl 4-(3-(2-fluoro-4-(methylsulfonyl)benzylamino)propylamino)piperidine-1-carboxylate (4 g, 9.02 mmol) in DMF (50 mL) was added di(1H-imidazol-1-yl)methanone (2.92 g, 18.0 mmol) and N-ethyl-N-isopropylpropan-2-amine (3.30 mL, 18.0 mmol) and the reaction was heated to 80° C. for overnight. The reaction was poured into water (200 mL) and extracted with EtOAc (400 mL). The organic layer was washed with 1N HCl (100 mL) and brine (100 mL), dried over MgSO₄, filtered and concentrated in vacuo. The crude material was chromatographed eluting with 70% EtOAc/Hexane to yield tert-butyl 4-(3-(2-fluoro-4-(methylsulfonyl)benzyl)-2-oxotetrahydropyrimidin-1(2H)-yl)piperidine-1-carboxylate (1.1 g, 2.34 mmol, 26.0% yield). Mass spectrum 370.2 (M+H-Boc).

### Intermediate 5

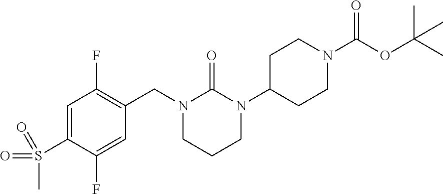 [SMILES: CC(C)(C)OC(=O)N1CCC(N2CCCN(Cc3cc(F)c(S(C)(=O)=O)cc3F)C2=O)CC1]

### tert-butyl 4-(3-(2,5-difluoro-4-(methylsulfonyl)benzyl)-2-oxotetrahydropyrimidin-1(2H)-yl)piperidine-1-carboxylate

Prepared according to the method described for Intermediate 4, substituting 4-bromo-2,5-difluorobenzaldehyde for 4-bromo-2,5-difluorobenzaldehyde in Step 1. Mass spectrum 388.1 (M+H-Boc).

### Intermediate 6

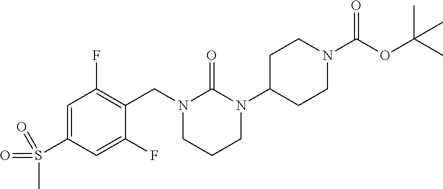 [SMILES: CC(C)(C)OC(=O)N1CCC(N2CCCN(Cc3c(F)cc(S(C)(=O)=O)cc3F)C2=O)CC1]

### tert-butyl 4-(3-(2,6-difluoro-4-(methylsulfonyl)benzyl)-2-oxotetrahydropyrimidin-1(2H)-yl)piperidine-1-carboxylate

Prepared according to the method described for Intermediate 4, substituting 4-bromo-2,5-difluorobenzaldehyde for 4-bromo-2,6-difluorobenzaldehyde in Step 1. Mass spectrum 387.8 (M+H-Boc).

### Intermediate 7

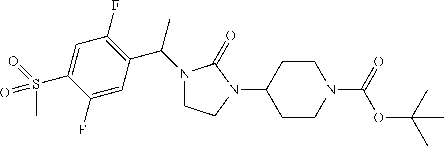 [SMILES: CC(c1cc(F)c(S(C)(=O)=O)cc1F)N1CCN(C2CCN(C(=O)OC(C)(C)C)CC2)C1=O]

### tert-butyl 4-(3-(1-(2,5-difluoro-4-(methylsulfonyl)phenyl)ethyl)-2-oxoimidazolidin-1-yl)piperidine-1-carboxylate

Step 1: To a solution of 1,4-dibromo-2,5-difluorobenzene (12.6 g, 46.3 mmol) in toluene (300 mL) cooled to −78° C. was added butyllithium (18.5 mL, 46.3 mmol) at such a rate that the internal temperature did not exceed −50° C. and the reaction was stirred for 45 minutes. To the reaction was added N, N-dimethylacetamide (4.84 g, 55.6 mmol). The reaction was stirred at −78° C. and then warmed to ambient temperature over a period of 4 hours. The reaction was quenched by adding water (300 mL) followed by the addition of EtOAc (500 mL) and the layers were separated. The organic layer was washed with brine (100 mL), dried over MgSO₄, filtered and concentrated in vacuo. The crude material was chromatographed eluting with 10% EtOAc/Hexane to give 1-(4-bromo-2,5-difluorophenyl)ethanone (5 g, 21.3 mmol, 45.9% yield).

Step 2: To a solution of tert-butyl 2-aminoethylcarbamate (1.2 g, 7.7 mmol) and 1-(4-bromo-2,5-difluorophenyl)ethanone (1.8 g, 7.7 mmol) in THF (50 mL) was added tetraisopropoxytitanium (2.6 g, 9.2 mmol) and the reaction was stirred overnight at ambient temperature. NaBH₄ (0.29 g, 7.7 mmol) was added and the reaction was stirred at ambient temperature for 3 hours. Water (100 mL) was added and aqueous layer was made basic with the addition of a saturated aqueous solution of NaHCO₃. To the reaction was added benzyl carbonochloridate (1.3 g, 7.7 mmol) and the reaction was stirred for 2 days at ambient temperature. The reaction was diluted with MTBE (250 mL) and the layers separated. The aqueous layer was again extracted with MTBE (100 mL). The combined organic layer was washed with brine (100 mL), dried over MgSO₄, filtered and concentrated in vacuo. The crude material was chromatographed eluting with 20% EtOAc/Hexane to yield benzyl(1-(4-bromo-2,5-difluorophenyl)ethyl)(2-((tert-butoxycarbonyl)amino)ethyl)carbamate (1.8 g, 46%).

Step 3: To a solution of benzyl(1-(4-bromo-2,5-difluorophenyl)ethyl)(2-((tert-butoxycarbonyl)amino)ethyl)carbamate (1.8 g, 3.5 mmol) in DCM (50 mL) was added 2,2,2-trifluoroacetic acid (4.0 g, 35 mmol) and the reaction was stirred at ambient temperature for 2 hours. The reaction was concentrated in vacuo to give benzyl 2-aminoethyl(1-(4-bromo-2,5-difluorophenyl)ethyl)carbamate 2,2,2-trifluoroacetate (1.8 g, 3.4 mmol, 97% yield).

Step 4: To a solution of benzyl 2-aminoethyl(1-(4-bromo-2,5-difluorophenyl)ethyl)carbamate 2,2,2-trifluoroacetate (1.8 g, 3.4 mmol) in DCE (100 mL) was added benzyl 4-oxopiperidine-1-carboxylate (1.2 g, 5.2 mmol) and the reaction was stirred at ambient temperature for 1 hour. To the reaction was added NaBH(OAc)₃ (1.4 g, 6.5 mmol) and the reaction was stirred overnight. The reaction was quenched by the addition of a saturated aqueous solution of NaHCO3 (100 mL) and then stirred at ambient temperature for 30 minutes. The layers were separated, the organic layer was concentrated in vacuo and the crude material was chromatographed eluting with 2% MeOH/EtOAc to give tert-butyl 4-(2-((benzyloxycarbonyl)(1-(4-bromo-2,5-difluorophenyl)ethyl)amino)ethylamino)piperidine-1-carboxylate (1.7 g, 2.8 mmol, 65% yield).

Step 5: To a solution of tert-butyl 4-(2-((benzyloxycarbonyl)(1-(4-bromo-2,5-difluorophenyl)ethyl)amino)ethylamino)piperidine-1-carboxylate (1.7 g, 2.8 mmol) in DMSO (20 mL) was added sodium methanesulfinate (0.44 g, 4.3 mmol) and cyclohexane-1,2-diamine (0.13 g, 1.1 mmol) and the reaction was degassed for 1 hour with nitrogen. Cu(I) triflate benzene complex (0.22 g, 0.43 mmol) was added and the reaction was heated at 110° C. overnight. The reaction was diluted with EtOAc (300 mL) and washed with NaOH (1M, 2×100 mL) and basic brine (50 mL), dried over Na₂SO₄, filtered and concentrated in vacuo. The crude material was chromatographed eluting with 5% MeOH/EtOAc to give tert-butyl 4-(2-((benzyloxycarbonyl)(1-(2,5-difluoro-4-(methylsulfonyl)phenyl)ethyl)amino)ethylamino)piperidine-1-carboxylate (0.7 g, 1.2 mmol, 41% yield).

Step 6: A solution of tert-butyl 4-(2-((benzyloxycarbonyl)(1-(2,5-difluoro-4-(methylsulfonyl)phenyl)ethyl)amino)ethylamino)piperidine-1-carboxylate (0.870 g, 1.46 mmol) in methanol (100 mL) was degassed with nitrogen. To the solution was added 200 mg of 10% Pd/C (Degussa type, 50% water) and the reaction was stirred overnight under a balloon of hydrogen. The reaction was purged with nitrogen and then filtered through Celite®. The solution was concentrated in vacuo and the crude material was taken up in DMF (40 mL). N-ethyl-N-isopropylpropan-2-amine (0.566 g, 4.38 mmol) and di(1H-imidazol-1-yl)methanone (0.710 g, 4.38 mmol) were added and the reaction was heated to 120° C. overnight. The reaction was poured into water (200 mL) and extracted with EtOAc (200 mL). The organic layer was washed with 1N HCl (100 mL) and brine (100 mL), dried over MgSO₄, filtered and concentrated in vacuo. The crude material was chromatographed eluting with 70% EtOAc/Hexane to yield (0.10 g, 0.205 mmol, 14.0% yield). Mass spectrum 388.1 (M+H-Boc).

### Intermediate 8

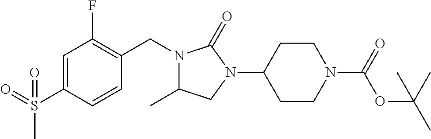 [SMILES: CC1CN(C2CCN(C(=O)OC(C)(C)C)CC2)C(=O)N1Cc1ccc(S(C)(=O)=O)cc1F]

### tert-butyl 4-(3-(2-fluoro-4-(methylsulfonyl)benzyl)-4-methyl-2-oxoimidazolidin-1-yl)piperidine-1-carboxylate

Prepared according to the method described for Intermediate 7, omitting step 1 and substituting 4-bromo-2-fluorobenzaldehyde for 1-(4-bromo-2,5-difluorophenyl)ethanone and tert-butyl 2-aminopropylcarbamate for tert-butyl 2-aminoethylcarbamate in Step 2. Mass spectrum 370.1 (M+H-Boc).

### Intermediate 9

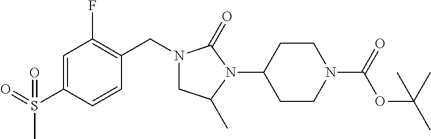 [SMILES: CC1CN(Cc2ccc(S(C)(=O)=O)cc2F)C(=O)N1C1CCN(C(=O)OC(C)(C)C)CC1]

### tert-butyl 4-(3-(2-fluoro-4-(methylsulfonyl)benzyl)-5-methyl-2-oxoimidazolidin-1-yl)piperidine-1-carboxylate

Step 1: To a solution of tert-butyl 1-aminopropan-2-ylcarbamate hydrochloride (5.0 g, 24 mmol) in MeOH (200 mL) was added sodium 2-methylpropan-2-olate (2.3 g, 24 mmol) in portions and the reaction was stirred at ambient temperature for 1 hour. To the reaction was added acetic acid (2.9 g, 47 mmol) followed by 4-bromo-2-fluorobenzaldehyde (4.8 g, 24 mmol) and the reaction was stirred at ambient temperature for 3 hours. The reaction was concentrated in vacuo and the residue taken up in DCE (400 mL). NaBH(OAc)₃ (7.5 g, 36 mmol) was added and the reaction was stirred for 3 days at ambient temperature. To the reaction was added a solution of NaHCO₃ (100 mL, saturated solution) followed by addition of benzyl carbonochloridate (4.0 g, 24 mmol) and the reaction was stirred at ambient temperature for 4 hours. The layers were separated and the organic layer was concentrated in vacuo. The crude material was chromatographed eluting with 10% EtOAc/Hexane to give benzyl 4-bromo-2-fluorobenzyl(2-((tert-butoxycarbonyl)amino) propyl)carbamate (8.8 g, 18 mmol, 75% yield).

Step 2: To a solution of benzyl 4-bromo-2-fluorobenzyl(2-((tert-butoxycarbonyl)amino)propyl)carbamate (8.8 g, 18 mmol) in DCM (100 mL) was added 2,2,2-trifluoroacetic acid (20 g, 178 mmol) and the reaction was stirred at ambient temperature for 3 hours. The reaction was concentrated in vacuo to give benzyl 2-aminopropyl(4-bromo-2-fluorobenzyl)carbamate 2,2,2-trifluoroacetate (9.0 g, 18 mmol, 99% yield).

Step 3: To a solution of benzyl 2-aminopropyl(4-bromo-2-fluorobenzyl)carbamate 2,2,2-trifluoroacetate (9.0 g, 18 mmol) in DCE (300 mL) was added tert-butyl 4-oxopiperidine-1-carboxylate (5.3 g, 27 mmol) and the reaction was stirred at ambient temperature for 2 hours. Na(OAc)₃BH (3.7 g, 18 mmol) was added and the reaction was stirred overnight at ambient temperature. To the reaction was added an additional 2.5 g of tert-butyl 4-oxopiperidine-1-carboxylate and 1.5 g of NaBH(OAc)₃ and the reaction was stirred for an additional 4 hours. The reaction was diluted with Na₂CO₃ (200 mL) and then stirred at ambient temperature for 1 hour. The organic layer was separated and concentrated in vacuo. The crude material was chromatographed eluting with a gradient of 50% EtOAc/DCM to 100% EtOAc to yield tert-butyl 4-(1-((benzyloxycarbonyl)(4-bromo-2-fluorobenzyl)amino)propan-2-ylamino)piperidine-1-carboxylate (9.5 g, 16 mmol, 93% yield).

Step 4: To a solution of tert-butyl 4-(1-((benzyloxycarbonyl)(4-bromo-2-fluorobenzyl)amino)propan-2-ylamino)piperidine-1-carboxylate (5.8 g, 10 mmol) in 100 mL DMSO was added cyclohexane-1,2-diamine (0.46 g, 4.0 mmol). Sodium methanesulfinate (1.5 g, 15 mmol) was added and the slurry was purged with N₂ for 1 hour. Cu(I) triflate benzene complex (0.50 g, 1.0 mmol) was added and the reaction was heated at 110° C. overnight. The reaction was diluted with EtOAc (400 mL), washed with NaOH (500 mL) and basic brine (150 mL), dried over Na₂SO⁴ and concentrated in vacuo. The crude material was chromatographed eluting with EtOAc to give tert-butyl 4-(1-((benzyloxycarbonyl)(2-fluoro-4-(methylsulfonyl)benzyl)amino)propan-2-ylamino)piperidine-1-carboxylate (2.5 g, 4.3 mmol, 43% yield).

Step 5: To a solution of tert-butyl 4-(1-((benzyloxycarbonyl)(2-fluoro-4-(methylsulfonyl)benzyl)amino)propan-2-ylamino)piperidine-1-carboxylate (2.5 g, 4.3 mmol) in EtOAc (300 mL) was added 1 g of 10% Pd/C (Degussa type, 50% water) and the reaction was stirred for 6 hours under an atmosphere of hydrogen. The reaction was filtered through Celite®, the filtrate was concentrated in vacuo and the residue was taken up in DMA (50 mL). Di(1H-imidazol-1-yl)methanone (1.4 g, 8.7 mmol) and Hunig's base (1.7 g, 13 mmol) were added and the reaction was heated to 100° C. for 6 hours. The reaction was diluted with EtOAc (400 mL), washed with water, 1N HCl (100 mL) and brine (100 mL), dried over MgSO₄, filtered and concentrated in vacuo. The crude material was chromatographed eluting with 1:1 Hexane/EtOAc to give tert-butyl 4-(3-(2-fluoro-4-(methylsulfonyl)benzyl)-5-methyl-2-oxoimidazolidin-1-yl)piperidine-1-carboxylate (0.800 g, 1.7 mmol, 39%). Mass spectrum 369.8 (M+H-Boc).

### Intermediate 10

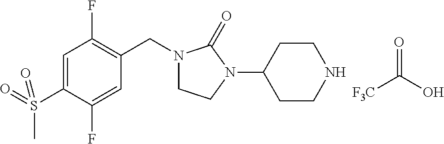

### 1-(2,5-difluoro-4-(methylsulfonyl)benzyl)-3-(piperidin-4-yl)imidazolidin-2-one 2,2,2-trifluoroacetate

To a solution of tert-butyl 4-(3-(2,5-difluoro-4-(methylsulfonyl)benzyl)-2-oxoimidazolidin-1-yl)piperidine-1-carboxylate (1.1 g, 2.3 mmol, Intermediate 1) in DCM (20 mL) was added TFA (10 mL) and the reaction was stirred at ambient temperature for 1 hour. The reaction was concentrated in vacuo to give 1-(2,5-difluoro-4-(methylsulfonyl)benzyl)-3-(piperidin-4-yl)imidazolidin-2-one 2,2,2-trifluoroacetate (1.1 g, 2.3 mmol, 97% yield).

The following deprotected intermediates were also prepared according to the method described for preparing Intermediate 10.

| Intermediate | | |
|---|---|---|
| # | Structure | Name |
| 11 | 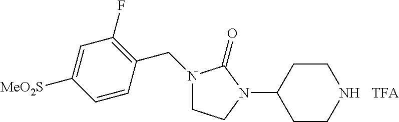 [SMILES: CS(=O)(=O)c1ccc(CN2CCN(C3CCNCC3)C2=O)c(F)c1 ; "TFA"] | 1-(2-fluoro-4- (methylsulfonyl)benzyl)-3- (piperidin-4-yl)imidazolidin-2- one 2,2,2-trifluoroacetate |
| 12 | 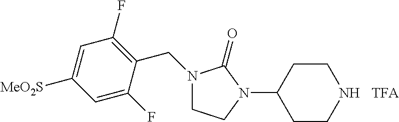 [SMILES: CS(=O)(=O)c1cc(F)c(CN2CCN(C3CCNCC3)C2=O)c(F)c1 ; "TFA"] | 1-(2,6-difluoro-4- (methylsulfonyl)benzyl)-3- (piperidin-4-yl)imidazolidin-2- one 2,2,2-trifluoroacetate |
| 13 | 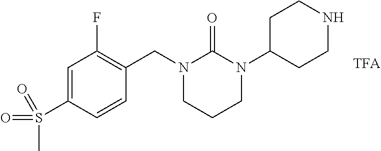 [SMILES: CS(=O)(=O)c1ccc(CN2CCCN(C3CCNCC3)C2=O)c(F)c1] | 1-(2-fluoro-4- (methylsulfonyl)benzyl)-3- (piperidin-4- yl)tetrahydropyrimidin-2(1H)- one 2,2,2-trifluoroacetate |
| 14 | 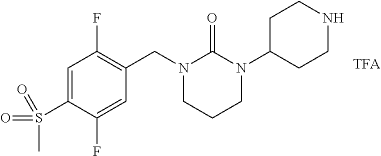 [SMILES: CS(=O)(=O)c1cc(F)c(CN2CCCN(C3CCNCC3)C2=O)cc1F] | 1-(2,5-difluoro-4- (methylsulfonyl)benzyl)-3- (piperidin-4- yl)tetrahydropyrimidin-2(1H)- one 2,2,2-trifluoroacetate |
| 15 | 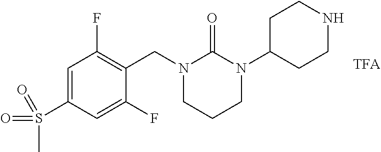 [SMILES: CS(=O)(=O)c1cc(F)c(CN2CCCN(C3CCNCC3)C2=O)c(F)c1] | 1-(2,6-difluoro-4- (methylsulfonyl)benzyl)-3- (piperidin-4- yl)tetrahydropyrimidin-2(1H)- one 2,2,2-trifluoroacetate |
| 16 | 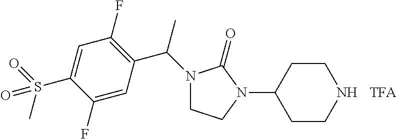 [SMILES: CC(c1cc(F)c(S(C)(=O)=O)cc1F)N1CCN(C2CCNCC2)C1=O ; "TFA"] | 1-(1-(2,5-difluoro-4- (methylsulfonyl)phenyl)ethyl)- 3-(piperidin-4-yl)imidazolidin- 2-one 2,2,2-trifluoroacetate |
| 17 | 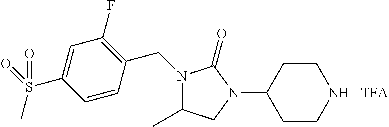 [SMILES: CC1CN(C2CCNCC2)C(=O)N1Cc1ccc(S(C)(=O)=O)cc1F ; "TFA"] | 3-(2-fluoro-4- (methylsulfonyl)benzyl)-4- methyl-1-(piperidin-4- yl)imidazolidin-2-one 2,2,2- trifluoroacetate |
| 18 | 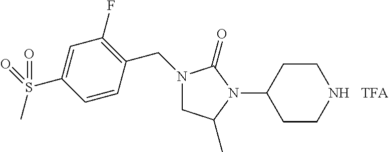 [SMILES: CC1CN(Cc2ccc(S(C)(=O)=O)cc2F)C(=O)N1C1CCNCC1 ; "TFA"] | 1-(2-fluoro-4- (methylsulfonyl)benzyl)-4- methyl-3-(piperidin-4- yl)imidazolidin-2-one 2,2,2- trifluoroacetate |

### Intermediate 19

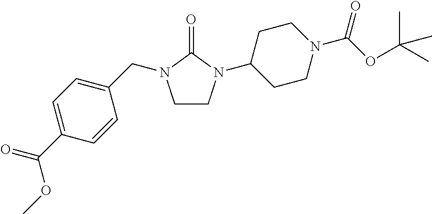 [SMILES: COC(=O)c1ccc(CN2CCN(C3CCN(C(=O)OC(C)(C)C)CC3)C2=O)cc1]

### tert-butyl 4-(3-(4-(methoxycarbonyl)benzyl)-2-oxoimidazolidin-1-yl)piperidine-1-carboxylate

Prepared according to the method for preparing Intermediate 1, omitting Step and Step 5 and substituting methyl 4-formyl benzoate for 4-bromo-2,5-difluorobenzaldehyde in Step 2. Mass spectrum (apci) m/z=318.2 (M+H-Boc).

### Intermediate 20

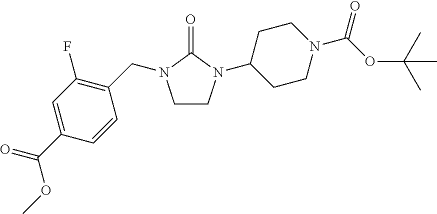 [SMILES: COC(=O)c1ccc(CN2CCN(C3CCN(C(=O)OC(C)(C)C)CC3)C2=O)c(F)c1]

### tert-butyl 4-(3-(2-fluoro-4-(methoxycarbonyl)benzyl)-2-oxoimidazolidin-1-yl)piperidine-1-carboxylate

Step 1: To a solution of 3-fluoro-4-formylbenzoic acid (0.350 g, 2.08 mmol) in DMF was added NaH (0.0916 g, 2.29 mmol). The mixture stirred for 30 minutes and iodomethane (0.325 g, 2.29 mmol) was added. This mixture stirred 5 hours. The mixture was poured into 1N HCl and extracted with ethyl acetate. The organic layer was washed with sodium bicarbonate and brine. The organics were dried over MgSO₄ and concentrated in vacuo. The crude material was purified by chromatography using Ethyl Acetate/Hexanes to give methyl 3-fluoro 4-formyl benzoate (0.25 g, 65.9%).

Step 2: Tert-butyl 4-(3-(2-fluoro-4-(methoxycarbonyl)benzyl)-2-oxoimidazolidin-1-yl)piperidine-1-carboxylate was prepared according to the method used to prepare Intermediate 1, omitting Step 1 and Step 5, and substituting methyl 3-fluoro 4-formyl benzoate for 4-bromo-2,5-difluorobenzaldehyde in Step 2. Mass spectrum (apci) m/z=336.2 (M+H-Boc).

### Intermediate 21

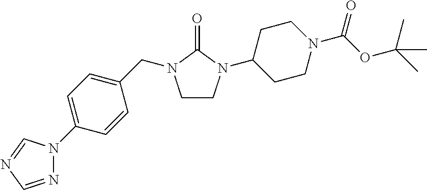 [SMILES: CC(C)(C)OC(=O)N1CCC(N2CCN(Cc3ccc(-n4cncn4)cc3)C2=O)CC1]

### tert-butyl 4-(3-(4-(1H-1,2,4-triazol-1-yl)benzyl)-2-oxoimidazolidin-1-yl)piperidine-1-carboxylate

Prepared according to the method for preparing Intermediate 1, omitting Step 1 and Step 5 and substituting 4-(1H-1,2,4-triazol-1-yl)benzaldehyde for 4-bromo-2,5-difluorobenzaldehyde in Step 2. Mass spectrum (apci) m/z=327.2 (M+H-Boc).

### Intermediate 22

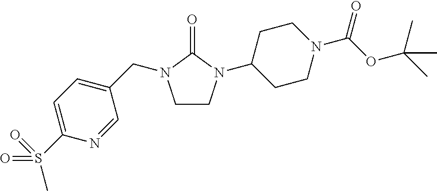 [SMILES: CC(C)(C)OC(=O)N1CCC(N2CCN(Cc3ccc(S(C)(=O)=O)nc3)C2=O)CC1]

### tert-butyl 4-(3-((6-(methylsulfonyl)pyridin-3-yl)methyl)-2-oxoimidazolidin-1-yl)piperidine-1-carboxylate

Prepared according to the method for preparing Intermediate 1, omitting Steps 1 and Steps 5 and substituting 6-(methylsulfonyl)nicotinaldehyde for 4-bromo-2,5-difluorobenzaldehyde in Step 2. Mass spectrum (apci) m/z=339.1 (M+H-Boc).

### Intermediate 23

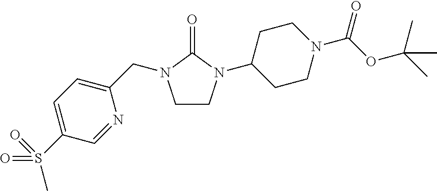 [SMILES: CC(C)(C)OC(=O)N1CCC(N2CCN(Cc3ccc(S(C)(=O)=O)cn3)C2=O)CC1]

### tert-butyl 4-(3-((5-(methylsulfonyl)pyridin-2-yl)methyl)-2-oxoimidazolidin-1-yl)piperidine-1-carboxylate

Prepared according to the method for preparing Intermediate 1, omitting Steps and Steps 5 and substituting 5-bromopicolinaldehyde for 4-bromo-2,5-difluorobenzaldehyde in Step 2. Mass spectrum (apci) m/z=339.1 (M+H-Boc).

### Intermediate 24

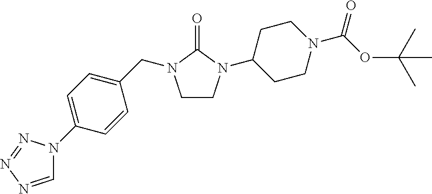 [SMILES: CC(C)(C)OC(=O)N1CCC(N2CCN(Cc3ccc(-n4cnnn4)cc3)C2=O)CC1]

### tert-butyl 4-(3-(4-(1H-tetrazol-1-yl)benzyl)-2-oxoimidazolidin-1-yl)piperidine-1-carboxylate

Step 1: To a slurry of 4-(1H-tetrazol-1-yl)benzoic acid (2.6 g, 14 mmol) in DCM (40 mL) was added Hunig's Base (5.5 g, 55 mmol) followed by benzotriazole-1-yl-oxy-tris-(dimethylamino)phosphonium hexafluorophosphate (5.2 g) and N,O-dimethylhydroxylamine hydrochloride (2.7 g, 27 mmol) and the reaction was stirred overnight at ambient temperature. The reaction was diluted with water and the layers were separated. The organic layer was concentrated in vacuo and the material chromatographed using 80:20 EtOAc/Hex as the eluent to give N-methoxy-N-methyl-4-(1H-tetrazol-1-yl)benzamide (1.7 g, 7.3 mmol, 53% yield).

Step 2: To a solution of N-methoxy-N-methyl-4-(1H-tetrazol-1-yl)benzamide (1.7 g, 7.3 mmol) in DCM (40 mL) cooled to 0° C. was added di-isobutyl aluminum chloride (1M solution in DCM) (11 mL, 11 mmol) and the reaction was stirred for 3 hours at ambient temperature. The reaction was diluted with water (50 mL) and the layers were separated. The organic layer was washed with brine (100 mL), dried over MgSO₄, filtered and concentrated in vacuo to provide 4-(1H-tetrazol-1-yl)benzaldehyde. The crude material was used immediately without further purification.

Step 3: Tert-butyl 4-(3-(4-(1H-tetrazol-1-yl)benzyl)-2-oxoimidazolidin-1-yl)piperidine-1-carboxylate was prepared according to the method for preparing Intermediate 1, omitting Step 1 and Step 5 and substituting 4-(1H-tetrazol-1-yl)benzaldehyde (synthesis below) for 4-bromo-2,5-difluorobenzaldehyde in Step 2. Mass spectrum (apci) m/z=336.2 (M+H-Boc).

The following deprotected intermediates were prepared from Intermediates 19-24 according to the method described for preparing Intermediate 10.

| Intermediate | | |
|---|---|---|
| # | Structure | Name |
| 25 | 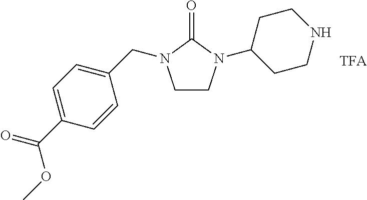 [SMILES: COC(=O)c1ccc(CN2CCN(C3CCNCC3)C2=O)cc1] | methyl 4-((2-oxo-3-(piperidin-4- yl)imidazolidin-1- yl)methyl)benzoate |
| 26 | 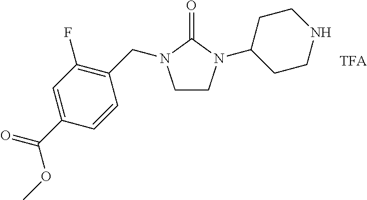 [SMILES: COC(=O)c1ccc(CN2CCN(C3CCNCC3)C2=O)c(F)c1] | methyl 3-fluoro-4-((2-oxo-3- (piperidin-4-yl)imidazolidin-1- yl)methyl)benzoate |
| 27 | 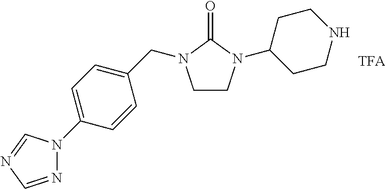 [SMILES: O=C1N(Cc2ccc(-n3cncn3)cc2)CCN1C1CCNCC1] | 1-(4-(1H-1,2,4-triazol-1- yl)benzyl)-3-(piperidin-4- yl)imidazolidin-2-one |
| 28 | 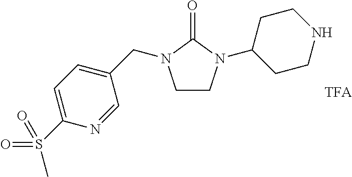 [SMILES: CS(=O)(=O)c1ccc(CN2CCN(C3CCNCC3)C2=O)cn1] | 1-((6-(methylsulfonyl)pyridin-3- yl)methyl)-3-(piperidin-4- yl)imidazolidin-2-one |
| 29 | 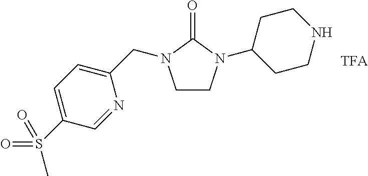 [SMILES: CS(=O)(=O)c1ccc(CN2CCN(C3CCNCC3)C2=O)nc1] | 1-((5-(methylsulfonyl)pyridin-2- yl)methyl)-3-(piperidin-4- yl)imidazolidin-2-one |
| 30 | 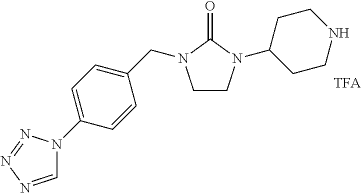 [SMILES: O=C1N(Cc2ccc(-n3cnnn3)cc2)CCN1C1CCNCC1] | 1-(4-(1H-tetrazol-1-yl)benzyl)-3- (piperidin-4-yl)imidazolidin-2-one |

### Example 1

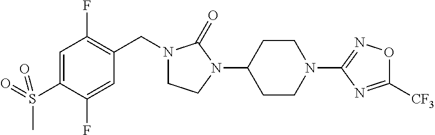 [SMILES: CS(=O)(=O)c1cc(F)c(CN2CCN(C3CCN(c4noc(C(F)(F)F)n4)CC3)C2=O)cc1F]

### 1-(2,5-difluoro-4-(methylsulfonyl)benzyl)-3-(1-(5-(trifluoromethyl)-1,2,4-oxadiazol-3-yl)piperidin-4-yl)imidazolidin-2-one

Step 1: To a solution of 1-(2,5-difluoro-4-(methylsulfonyl)benzyl)-3-(piperidin-4-yl)imidazolidin-2-one 2,2,2-trifluoroacetate (Intermediate 10; 0.50 g, 1.0 mmol) in THF (30 mL) was added NaHCO₃ (saturated aqueous solution, 30 mL) followed by cyanic bromide (0.21 mL, 1.0 mmol, 5M solution in ACN) and the reaction was stirred for 2 hours at ambient temperature. The reaction was diluted with EtOAc (200 mL) and the layers separated. The organic layer was washed with brine (50 mL), dried over MgSO₄, filtered and concentrated in vacuo to give 4-(3-(2,5-difluoro-4-(methylsulfonyl)benzyl)-2-oxoimidazolidin-1-yl)piperidine-1-carbonitrile (0.22 g, 0.55 mmol, 54% yield).

Step 2: To a solution of 4-(3-(2,5-difluoro-4-(methylsulfonyl)benzyl)-2-oxoimidazolidin-1-yl)piperidine-1-carbonitrile (1.9 g, 4.8 mmol) in THF (20 mL) was added hydroxylamine (0.63 g, 9.5 mmol, 50% solution in water) and the reaction was heated to 60° C. for 4 hours. The reaction was concentrated in vacuo and the residue was dissolved in THF (40 mL). 2,2,2-Trifluoroacetic anhydride (1.0 mL, 7.0 mmol) was added and the reaction was stirred overnight at ambient temperature. The reaction was concentrated in vacuo, partitioned between EtOAc (200 mL) and a saturated solution of NaHCO₃ (50 mL), and the biphasic mixture stirred for 30 minutes. The layers were separated and the organic layer was washed with brine (100 mL), dried over MgSO₄, filtered and concentrated in vacuo. The crude material was chromatographed eluting with 70% EtOAc/Hexane. The crude material was taken up in IPA (4 mL) and the solution was added to 40 mL of water, at which point a solid formed. The slurry was stirred for 2 hours and filtered. The solid was dried on the filter for 30 minutes, then dried in vacuo give 1-(2,5-difluoro-4-(methylsulfonyl)benzyl)-3-(1-(5-(trifluoromethyl)-1,2,4-oxadiazol-3-yl)piperidin-4-yl)imidazolidin-2-one (1.1 g, 2.2 mmol, 47% yield). Mass spectrum 509.7 (M+H).

The following compounds made according to the procedure of Example 1.

| | | | MS data |
|---|---|---|---|
| Ex. # | Compound | Name | (apci) m/z |
| 2 | 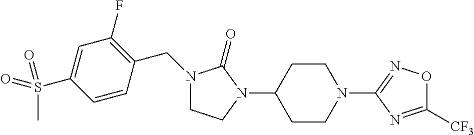 [SMILES: CS(=O)(=O)c1ccc(CN2CCN(C3CCN(c4noc(C(F)(F)F)n4)CC3)C2=O)c(F)c1] | 1-(2-fluoro-4- (methylsulfonyl)benzyl)-3- (1-(5-(trifluoromethyl)-1,2,4- oxadiazol-3-yl)piperidin-4- yl)imidazolidin-2-one | 492.0 (M + H) |
| 3 | 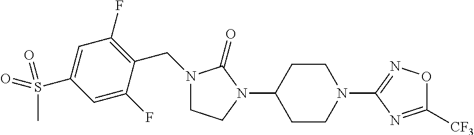 [SMILES: CS(=O)(=O)c1cc(F)c(CN2CCN(C3CCN(c4noc(C(F)(F)F)n4)CC3)C2=O)c(F)c1] | 1-(2,6-difluoro-4- (methylsulfonyl)benzyl)-3- (1-(5-(trifluoromethyl)-1,2,4- oxadiazol-3-yl)piperidin-4- yl)imidazolidin-2-one | 510.1 (M + H) |
| 4 | 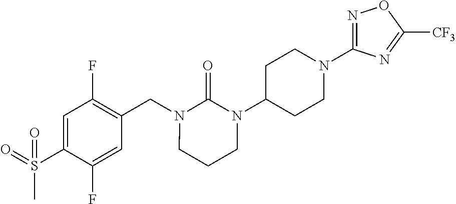 [SMILES: CS(=O)(=O)c1cc(F)c(CN2CCCN(C3CCN(c4noc(C(F)(F)F)n4)CC3)C2=O)cc1F] | 1-(2,5-difluoro-4- (methylsulfonyl)benzyl)-3- (1-(5-(trifluoromethyl)-1,2,4- oxadiazol-3-yl)piperidin-4- yl)tetrahydropyrimidin- 2(1H)-one | 523.7 (M + H) |
| 5 | 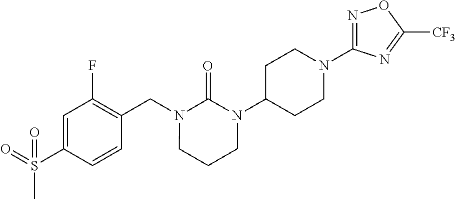 [SMILES: CS(=O)(=O)c1ccc(CN2CCCN(C3CCN(c4noc(C(F)(F)F)n4)CC3)C2=O)c(F)c1] | 1-(2-fluoro-4- (methylsulfonyl)benzyl)-3- (1-(5-(trifluoromethyl)-1,2,4- oxadiazol-3-yl)piperidin-4- yl)tetrahydropyrimidin- 2(1H)-one | 506.1 (M + H) |
| 6 | 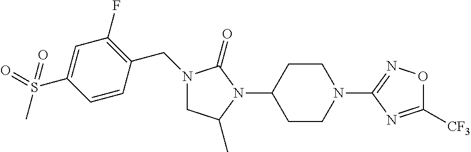 [SMILES: CC1CN(Cc2ccc(S(C)(=O)=O)cc2F)C(=O)N1C1CCN(c2noc(C(F)(F)F)n2)CC1] | 1-(2-fluoro-4- (methylsulfonyl)benzyl)-4- methyl-3-(1-(5- (trifluoromethyl)-1,2,4- oxadiazol-3-yl)piperidin-4- yl)imidazolidin-2-one | 505.7 (M + H) |

### Example 7

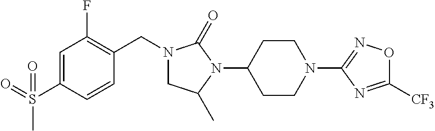 [SMILES: CC1CN(Cc2ccc(S(C)(=O)=O)cc2F)C(=O)N1C1CCN(c2noc(C(F)(F)F)n2)CC1]

### Enantiomer 1 of 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one

Racemic 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one (Example 6) was separated into its enantiomers using a Chiral Technologies, Inc. 2.1 cm×250 mm column with 5 micron OJ-H packing, eluting with 30% ethanol/70% hexanes at a flow rate of 21 mL/min. and using 220 nm wavelength as the detection wavelength. The first peak to elute was collected and designated “Enantiomer 1” of 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one. Mass spectrum 505.7 (M+H).

### Example 8

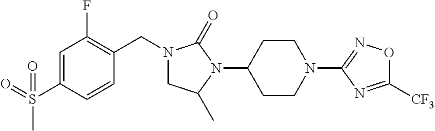 [SMILES: CC1CN(Cc2ccc(S(C)(=O)=O)cc2F)C(=O)N1C1CCN(c2noc(C(F)(F)F)n2)CC1]

### Enantiomer 2 of 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one

Racemic 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one (Example 6) was separated into its enantiomers using a Chiral Technologies, Inc. 2.1 cm×250 mm column with 5 micron OJ-H packing eluting with 30% ethanol/70% hexanes with a flow rate of 21 mL/min. and using 220 nm wavelength as the detection wavelength. The second peak to elute was collected and designated “Enantiomer 2” of 1-(2-fluoro-4-(methylsulfonyl)benzyl)-4-methyl-3-(1-(5-(trifluoromethyl)-1,2,4-oxadiazol-3-yl)piperidin-4-yl)imidazolidin-2-one. Mass spectrum 505.7 (M+H).

### Example 9

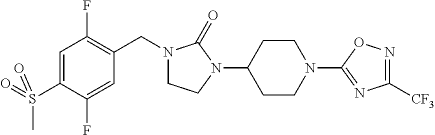 [SMILES: CS(=O)(=O)c1cc(F)c(CN2CCN(C3CCN(c4nc(C(F)(F)F)no4)CC3)C2=O)cc1F]

### 1-(2,5-difluoro-4-(methylsulfonyl)benzyl)-3-(1-(3-(trifluoromethyl)-1,2,4-oxadiazol-5-yl)piperidin-4-yl)imidazolidin-2-one

To a solution of 4-(3-(2,5-difluoro-4-(methylsulfonyl)benzyl)-2-oxoimidazolidin-1-yl)piperidine-1-carbonitrile (Example 1, Step A; 0.22 g, 0.552 mmol) in dioxanes (2 mL) was added 2,2,2-trifluoro-N′-hydroxyacetamidine (prepared as described in Example 7 of PCT Publication No. WO 2006/044958, which is incorporated herein by reference) (0.141 g, 1.10 mmol) followed by zinc (II) chloride (1.99 mL, 0.994 mmol) and the reaction was heated to 80° C. in a sealed tube overnight. The reaction was diluted with EtOAc (100 mL) and stirred with a saturated solution of NaHCO₃ (20 mL) for 20 minutes, and the layers were separated. The organic layer was washed with water (30 mL) and brine (30 mL), dried over MgSO₄, filtered and concentrated in vacuo. The crude material was chromatographed eluting with 70% EtOAc/Hexane to yield a solid, which was triturated with ether to give 1-(2,5-difluoro-4-(methylsulfonyl)benzyl)-3-(1-(3-(trifluoromethyl)-1,2,4-oxadiazol-5-yl)piperidin-4-yl)imidazolidin-2-one (0.0287 g, 0.0563 mmol, 10.2% yield). Mass spectrum 510.0 (M+H).

### Example 10

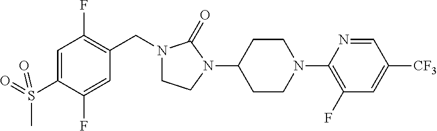 [SMILES: CS(=O)(=O)c1cc(F)c(CN2CCN(C3CCN(c4ncc(C(F)(F)F)cc4F)CC3)C2=O)cc1F]

### 1-(2,5-difluoro-4-(methylsulfonyl)benzyl)-3-(1-(3-fluoro-5-(trifluoromethyl)pyridin-2-yl)piperidin-4-yl)imidazolidin-2-one

To a solution of 1-(2,5-difluoro-4-(methylsulfonyl)benzyl)-3-(piperidin-4-yl)imidazolidin-2-one 2,2,2-trifluoroacetate (Intermediate 10; 0.10 g, 0.27 mmol) in DMF (2 mL) was added diisopropylethylamine (0.10 g, 0.80 mmol) and 2,3-difluoro-5-(trifluoromethyl)pyridine (0.049 g, 0.27 mmol) and the reaction was stirred at ambient temperature for 3 hours. The reaction was diluted with EtOAc (50 mL), washed with water (50 mL) and brine (50 mL), dried over MgSO₄, filtered and concentrated in vacuo. The crude material was chromatographed eluting with 20% DCM/EtOAc to yield 1-(2,5-difluoro-4-(methylsulfonyl)benzyl)-3-(1-(3-fluoro-5-(trifluoromethyl)pyridin-2-yl)piperidin-4-yl)imidazolidin-2-one (0.023 g, 0.043 mmol, 16% yield). Mass spectrum 537.2 (M+H).

The following compounds were made using a similar procedure as described for Example 10.

| | | | MS data |
|---|---|---|---|
| Ex. # | Compound | Name | (apci) m/z |
| 11 | 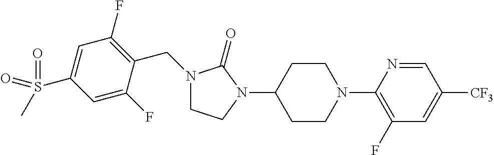 [SMILES: CS(=O)(=O)c1cc(F)c(CN2CCN(C3CCN(c4ncc(C(F)(F)F)cc4F)CC3)C2=O)c(F)c1] | 1-(2,6-difluoro-4- (methylsulfonyl)benzyl)-3- (1-(3-fluoro-5- (trifluoromethyl)pyridin-2- yl)piperidin-4- yl)imidazolidin-2-one | 537.2 (M + H) |
| 12 | 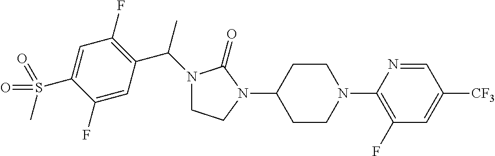 [SMILES: CC(c1cc(F)c(S(C)(=O)=O)cc1F)N1CCN(C2CCN(c3ncc(C(F)(F)F)cc3F)CC2)C1=O] | 1-(1-(2,5-difluoro-4- (methylsulfonyl)phenyl) ethyl)-3-(1-(3-fluoro-5- (trifluoromethyl)pyridin-2- yl)piperidin-4- yl)imidazolidin-2-one | 551.2 (M + H) |
| 13 | 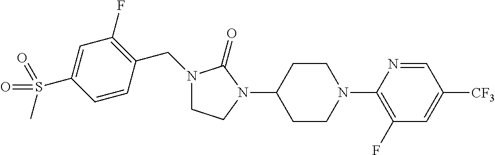 [SMILES: CS(=O)(=O)c1ccc(CN2CCN(C3CCN(c4ncc(C(F)(F)F)cc4F)CC3)C2=O)c(F)c1] | 1-(2-fluoro-4- (methylsulfonyl)benzyl)-3- (1-(3-fluoro-5- (trifluoromethyl)pyridin-2- yl)piperidin-4- yl)imidazolidin-2-one | 519.1 (M + H) |
| 14 | 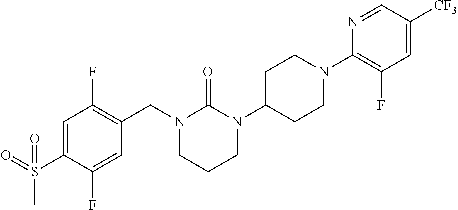 [SMILES: CS(=O)(=O)c1cc(F)c(CN2CCCN(C3CCN(c4ncc(C(F)(F)F)cc4F)CC3)C2=O)cc1F] | 1-(2,5-difluoro-4- (methylsulfonyl)benzyl)-3- (1-(3-fluoro-5- (trifluoromethyl)pyridin-2- yl)piperidin-4- yl)tetrahydropyrimidin- 2(1H)-one | 551.2 (M + H) |
| 15 | 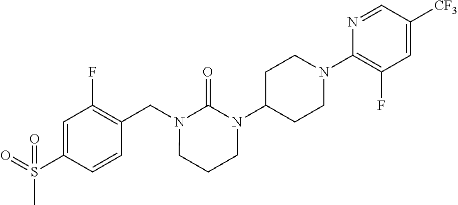 [SMILES: CS(=O)(=O)c1ccc(CN2CCCN(C3CCN(c4ncc(C(F)(F)F)cc4F)CC3)C2=O)c(F)c1] | 1-(2-fluoro-4- (methylsulfonyl)benzyl)-3- (1-(3-fluoro-5- (trifluoromethyl)pyridin-2- yl)piperidin-4- yl)tetrahydropyrimidin- 2(1H)-one | 533.2 (M + H) |
| 16 | 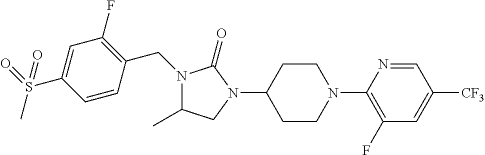 [SMILES: CC1CN(C2CCN(c3ncc(C(F)(F)F)cc3F)CC2)C(=O)N1Cc1ccc(S(C)(=O)=O)cc1F] | 3-(2-fluoro-4- (methylsulfonyl)benzyl)-1- (1-(3-fluoro-5- (trifluoromethyl)pyridin-2- yl)piperidin-4-yl)-4- methylimidazolidin-2-one | 533.2 (M + H) |
| 17 | 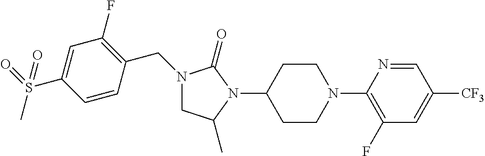 [SMILES: CC1CN(Cc2ccc(S(C)(=O)=O)cc2F)C(=O)N1C1CCN(c2ncc(C(F)(F)F)cc2F)CC1] | 1-(2-fluoro-4- (methylsulfonyl)benzyl)-3- (1-(3-fluoro-5- (trifluoromethyl)pyridin-2- yl)piperidin-4-yl)-4- methylimidazolidin-2-one | 532.7 (M + H) |

### Example 18

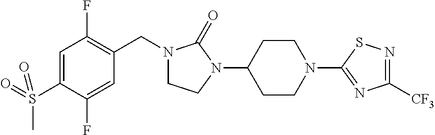 [SMILES: CS(=O)(=O)c1cc(F)c(CN2CCN(C3CCN(c4nc(C(F)(F)F)ns4)CC3)C2=O)cc1F]

### 1-(2,5-difluoro-4-(methylsulfonyl)benzyl)-3-(1-(3-(trifluoromethyl)-1,2,4-thiadiazol-5-yl)piperidin-4-yl)imidazolidin-2-one

To a solution of 1-(2,5-difluoro-4-(methylsulfonyl)benzyl)-3-(piperidin-4-yl)imidazolidin-2-one 2,2,2-trifluoroacetate (Intermediate 10; 0.10 g, 0.27 mmol) in DMF (2 mL) at 0° C. was added N-ethyl-N-isopropylpropan-2-amine (0.10 g, 0.80 mmol) followed by 5-chloro-3-(trifluoromethyl)-1,2,4-thiadiazole (0.050 g, 0.27 mmol) and the reaction was stirred for 3 hours while warming to ambient temperature. The reaction was diluted with EtOAc (100 mL), washed with water (50 mL), brine (50 mL), dried over MgSO₄, filtered and concentrated in vacuo. The crude material was chromatographed eluting with 20% DCM/EtOAc to yield 1-(2,5-difluoro-4-(methylsulfonyl)benzyl)-3-(1-(3-(trifluoromethyl)-1,2,4-thiadiazol-5-yl)piperidin-4-yl)imidazolidin-2-one (0.025 g, 0.048 mmol, 18% yield). Mass spectrum 526.1 (M+H).

The following compounds were made using a similar procedure as described for Example 18.

| | | | MS data |
|---|---|---|---|
| Ex. # | Compound | Name | (apci) m/z |
| 19 | 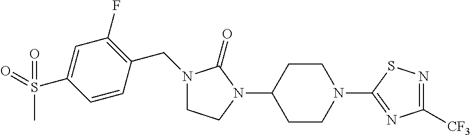 [SMILES: CS(=O)(=O)c1ccc(CN2CCN(C3CCN(c4nc(C(F)(F)F)ns4)CC3)C2=O)c(F)c1] | 1-(2-fluoro-4- (methylsulfonyl)benzyl)- 3-(1-(3- (trifluoromethyl)-1,2,4- thiadiazol-5- yl)piperidin-4- yl)imidazolidin-2-one | 508.1 (M + H) |
| 20 | 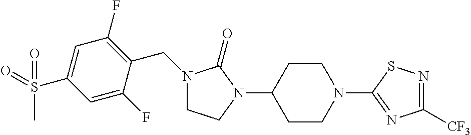 [SMILES: CS(=O)(=O)c1cc(F)c(CN2CCN(C3CCN(c4nc(C(F)(F)F)ns4)CC3)C2=O)c(F)c1] | 1-(2,6-difluoro-4- (methylsulfonyl)benzyl)- 3-(1-(3- (trifluoromethyl)-1,2,4- thiadiazol-5- yl)piperidin-4- yl)imidazolidin-2-one | 526.1 (M + H) |
| 21 | 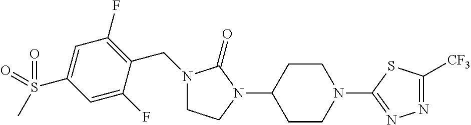 [SMILES: CS(=O)(=O)c1cc(F)c(CN2CCN(C3CCN(c4nnc(C(F)(F)F)s4)CC3)C2=O)c(F)c1] | 1-(2,6-difluoro-4- (methylsulfonyl)benzyl)- 3-(1-(5- (trifluoromethyl)-1,3,4- thiadiazol-2- yl)piperidin-4- yl)imidazolidin-2-one | 526.1 (M + H) |
| 22 | 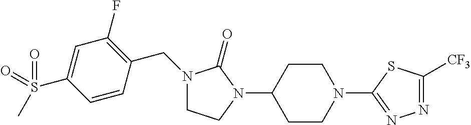 [SMILES: CS(=O)(=O)c1ccc(CN2CCN(C3CCN(c4nnc(C(F)(F)F)s4)CC3)C2=O)c(F)c1] | 1-(2-fluoro-4- (methylsulfonyl)benzyl)- 3-(1-(5- (trifluoromethyl)-1,3,4- thiadiazol-2- yl)piperidin-4- yl)imidazolidin-2-one | 508.1 (M + H) |
| 23 | 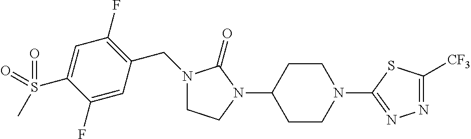 [SMILES: CS(=O)(=O)c1cc(F)c(CN2CCN(C3CCN(c4nnc(C(F)(F)F)s4)CC3)C2=O)cc1F] | 1-(2,5-difluoro-4- (methylsulfonyl)benzyl)- 3-(1-(5- (trifluoromethyl)-1,3,4- thiadiazol-2- yl)piperidin-4- yl)imidazolidin-2-one | 526.1 (M + H) |
| 24 | 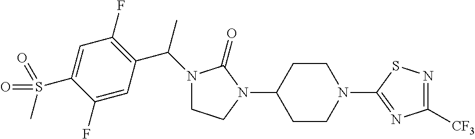 [SMILES: CC(c1cc(F)c(S(C)(=O)=O)cc1F)N1CCN(C2CCN(c3nc(C(F)(F)F)ns3)CC2)C1=O] | 1-(1-(2,5-difluoro-4- (methylsulfonyl)phenyl) ethyl)-3-(1-(3- (trifluoromethyl)-1,2,4- thiadiazol-5- yl)piperidin-4- yl)imidazolidin-2-one | 540.1 (M + H) |
| 25 | 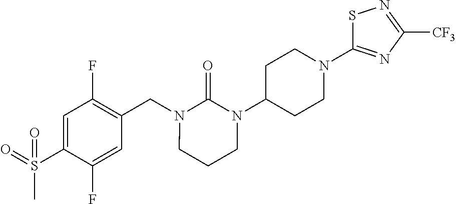 [SMILES: CS(=O)(=O)c1cc(F)c(CN2CCCN(C3CCN(c4nc(C(F)(F)F)ns4)CC3)C2=O)cc1F] | 1-(2,5-difluoro-4- (methylsulfonyl)benzyl)- 3-(1-(3- (trifluoromethyl)-1,2,4- thiadiazol-5- yl)piperidin-4- yl)tetrahydropyrimidin- 2(1H)-one | 540.1 (M + H) |
| 26 | 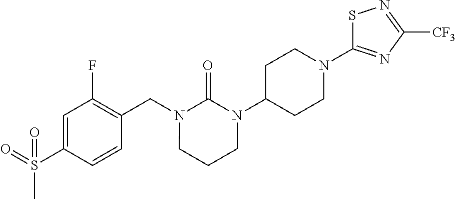 [SMILES: CS(=O)(=O)c1ccc(CN2CCCN(C3CCN(c4nc(C(F)(F)F)ns4)CC3)C2=O)c(F)c1] | 1-(2-fluoro-4- (methylsulfonyl)benzyl)- 3-(1-(3- (trifluoromethyl)-1,2,4- thiadiazol-5- yl)piperidin-4- yl)tetrahydropyrimidin- 2(1H)-one | 522.1 (M + H) |
| 27 | 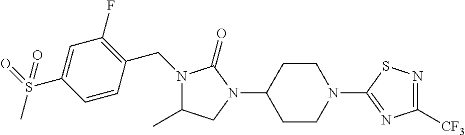 [SMILES: CC1CN(C2CCN(c3nc(C(F)(F)F)ns3)CC2)C(=O)N1Cc1ccc(S(C)(=O)=O)cc1F] | 3-(2-fluoro-4- (methylsulfonyl)benzyl)- 4-methyl-1-(1-(3- (trifluoromethyl)-1,2,4- thiadiazol-5- yl)piperidin-4- yl)imidazolidin-2-one | 522.1 (M + H) |
| 28 | 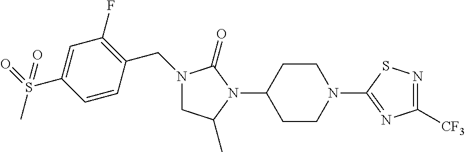 [SMILES: CC1CN(Cc2ccc(S(C)(=O)=O)cc2F)C(=O)N1C1CCN(c2nc(C(F)(F)F)ns2)CC1] | 1-(2-fluoro-4- (methylsulfonyl)benzyl)- 4-methyl-3-(1-(3- (trifluoromethyl)-1,2,4- thiadiazol-5- yl)piperidin-4- yl)imidazolidin-2-one | 521.7 (M + H) |

### Example 29

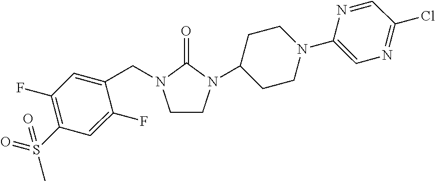 [SMILES: CS(=O)(=O)c1cc(F)c(CN2CCN(C3CCN(c4cnc(Cl)cn4)CC3)C2=O)cc1F]

### 1-(1-(5-chloropyrazin-2-yl)piperidin-4-yl)-3-(2,5-difluoro-4-(methylsulfonyl)benzyl)imidazolidin-2-one

To a solution of 1-(2,5-difluoro-4-(methylsulfonyl)benzyl)-3-(piperidin-4-yl)imidazolidin-2-one 2,2,2-trifluoroacetate (Intermediate 10; 0.2 g, 0.410 mmol) in DMF (2 mL) was added N-ethyl-N-isopropylpropan-2-amine (0.300 mL, 1.641 mmol) and 2,5-dichloropyrazine (0.0917 g, 0.615 mmol) and the reaction was heated to 100° C. for 8 hours. The reaction was diluted with EtOAc (100 mL), washed with water (50 mL) and brine (50 mL), dried over MgSO₄, filtered and concentrated in vacuo. The crude material was chromatographed eluting with 7:3 EtOAc/Hexane to yield a solid, which was further purified by reverse phase preparative HPLC to give 1-(1-(5-chloropyrazin-2-yl)piperidin-4-yl)-3-(2,5-difluoro-4-(methylsulfonyl)benzyl)imidazolidin-2-one (0.0071 g, 0.0146 mmol, 3.6% yield). Mass spectrum 485.7, 487.7 (M+H).

The following compounds were made using a similar procedure as described for Example 29.

| | | | MS data |
|---|---|---|---|
| Ex. # | Compound | Name | (apci) m/z |
| 30 | 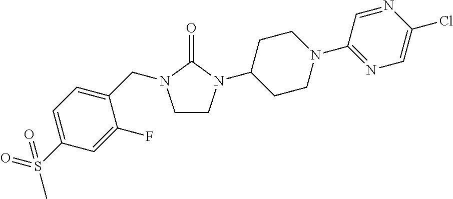 [SMILES: CS(=O)(=O)c1ccc(CN2CCN(C3CCN(c4cnc(Cl)cn4)CC3)C2=O)c(F)c1] | 1-(1-(5-chloropyrazin-2- yl)piperidin-4-yl)-3-(2- fluoro-4- (methylsulfonyl)benzyl) imidazolidin-2-one | 468.1, 470.2 (M + H) |
| 31 | 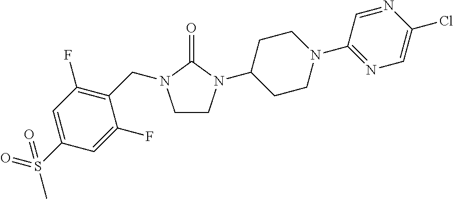 [SMILES: CS(=O)(=O)c1cc(F)c(CN2CCN(C3CCN(c4cnc(Cl)cn4)CC3)C2=O)c(F)c1] | 1-(1-(5-chloropyrazin-2- yl)piperidin-4-yl)-3-(2,6- difluoro-4- (methylsulfonyl)benzyl) imidazolidin-2-one | 486.0, 488.0 (M + H) |

### Example 32

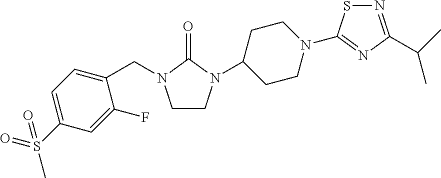 [SMILES: CC(C)c1nsc(N2CCC(N3CCN(Cc4ccc(S(C)(=O)=O)cc4F)C3=O)CC2)n1]

### 1-(2-fluoro-4-(methylsulfonyl)benzyl)-3-(1-(3-isopropyl-1,2,4-thiadiazol-5-yl)piperidin-4-yl)imidazolidin-2-one

To a solution of 1-(2-fluoro-4-(methylsulfonyl)benzyl)-3-(piperidin-4-yl)imidazolidin-2-one 2,2,2-trifluoroacetate (Intermediate 11; 100 mg, 0.213 mmol) in DMF (2 mL) was added N-ethyl-N-isopropylpropan-2-amine (111 μL, 0.639 mmol) and 5-chloro-3-isopropyl-1,2,4-thiadiazole (41.6 mg, 0.256 mmol) and the reaction was heated to 100° C. for 1 hour. The reaction was cooled and diluted with water (20 mL). The aqueous layer was extracted with EtOAc (50 mL), washed with water (20 mL) and brine (20 mL), dried over MgSO₄, filtered and concentrated in vacuo. The residue was chromatographed eluting with EtOAc to afford 1-(2-fluoro-4-(methylsulfonyl)benzyl)-3-(1-(3-isopropyl-1,2,4-thiadiazol-5-yl)piperidin-4-yl)imidazolidin-2-one (38.6 mg, 0.0801 mmol, 37.6% yield) as a yellow solid. Mass spectrum 480.2, 482.1 (M+H).

### Example 33

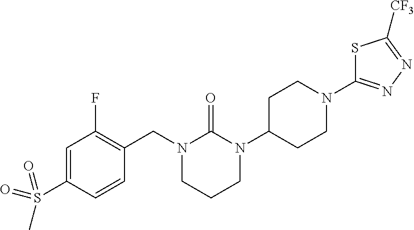 [SMILES: CS(=O)(=O)c1ccc(CN2CCCN(C3CCN(c4nnc(C(F)(F)F)s4)CC3)C2=O)c(F)c1]

### 1-(2-fluoro-4-(methylsulfonyl)benzyl)-3-(1-(5-(trifluoromethyl)-1,3,4-thiadiazol-2-yl)piperidin-4-yl)tetrahydropyrimidin-2(1H)-one

Prepared according to the method of Example 18. Mass spectrum (apci) m/z=522.1 (M+H).

### Example 34

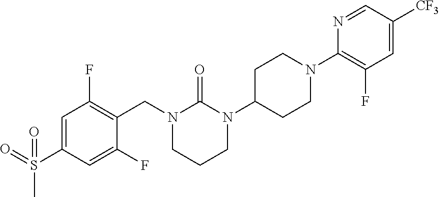 [SMILES: CS(=O)(=O)c1cc(F)c(CN2CCCN(C3CCN(c4ncc(C(F)(F)F)cc4F)CC3)C2=O)c(F)c1]

### 1-(2,6-difluoro-4-(methylsulfonyl)benzyl)-3-(1-(3-fluoro-5-(trifluoromethyl)pyridin-2-yl)piperidin-4-yl)tetrahydropyrimidin-2(1H)-one

Prepared according to the method of Example 10. Mass spectrum (apci) m/z=550.7 (M+H).

### Example 35

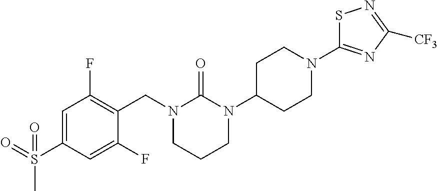 [SMILES: CS(=O)(=O)c1cc(F)c(CN2CCCN(C3CCN(c4nc(C(F)(F)F)ns4)CC3)C2=O)c(F)c1]

### 1-(2,6-difluoro-4-(methylsulfonyl)benzyl)-3-(1-(3-(trifluoromethyl)-1,2,4-thiadiazol-5-yl)piperidin-4-yl)tetrahydropyrimidin-2(1H)-one

Prepared according to the method of Example 18. Mass spectrum (apci) m/z=539.7 (M+H).

### Example 36

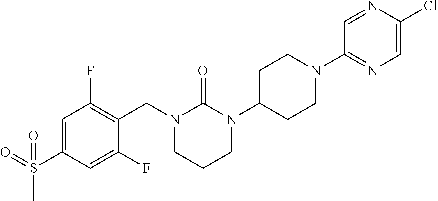 [SMILES: CS(=O)(=O)c1cc(F)c(CN2CCCN(C3CCN(c4cnc(Cl)cn4)CC3)C2=O)c(F)c1]

### 1-(1-(5-chloropyrazin-2-yl)piperidin-4-yl)-3-(2,6-difluoro-4-(methylsulfonyl)benzyl)tetrahydropyrimidin-2(1H)-one

Prepared according to the method of Example 29. Mass Spectrum (apci) m/z=499.7, 501.8 (M+H).

### Example 37

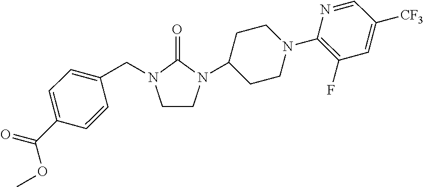 [SMILES: COC(=O)c1ccc(CN2CCN(C3CCN(c4ncc(C(F)(F)F)cc4F)CC3)C2=O)cc1]

### methyl 4-((3-(1-(3-fluoro-5-(trifluoromethyl)pyridin-2-yl)piperidin-4-yl)-2-oxoimidazolidin-1-yl)methyl)benzoate

Prepared according to the method of Example 1 from Intermediate 25. Mass spectrum (apci) m/z=m/z=481.2 (M+H).

### Example 38

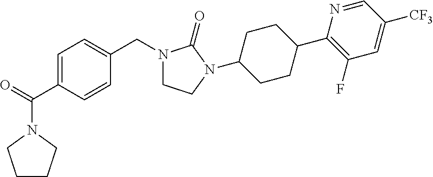 [SMILES: O=C(c1ccc(CN2CCN(C3CCC(c4ncc(C(F)(F)F)cc4F)CC3)C2=O)cc1)N1CCCC1]

### 1-(1-(3-fluoro-5-(trifluoromethyl)pyridin-2-yl)piperidin-4-yl)-3-(4-(pyrrolidine-1-carbonyl)benzyl)imidazolidin-2-one

Step 1: To a solution of methyl 4-((3-(1-(3-fluoro-5-(trifluoromethyl)pyridin-2-yl)piperidin-4-yl)-2-oxoimidazolidin-1-yl)methyl)benzoate (Example 37; 3.2 g, 6.7 mmol) in THF (50 mL) was added lithium hydroxide (27 mL, 27 mmol) (1M in water) and the reaction was stirred at ambient temperature for 3 hours. The reaction was diluted with water and the aqueous layer washed with ether. The aqueous layer was acidified with 1N HCl to a pH 2 and extracted with EtOAc (200 mL). The organic layer was washed with brine (50 mL), dried over MgSO4, filtered and concentrated in vacuo. The solid was triturated with ether to give 4-((3-(1-(3-fluoro-5-(trifluoromethyl)pyridin-2-yl)piperidin-4-yl)-2-oxoimidazolidin-1-yl)methyl)benzoic acid (2 g, 64%).

Step 2: 4-((3-(1-(3-Fluoro-5-(trifluoromethyl)pyridin-2-yl)piperidin-4-yl)-2-oxoimidazolidin-1-yl)methyl)benzoic acid (0.100 g, 0.214 mmol), HATU (0.0815 g, 0.214 mmol), and triethylamine (0.0448 mL, 0.322 mmol) were dissolved in DMF (1 mL) and the solution was stirred at ambient temperature for 30 minutes. Pyrrolidine (0.0305 g, 0.429 mmol) was added and the mixture was stirred for 2 hours. The solution was poured into water (10 mL) and extracted with ethyl acetate (3×10 mL). The organic layer was washed with brine, dried over MgSO4 and filtered. The organic layer was concentrated in vacuo and the crude product was purified by column chromatography using 25-75% EtOAc/Hexanes as the eluent to give 1-(1-(3-fluoro-5-(trifluoromethyl)pyridin-2-yl)piperidin-4-yl)-3-(4-(pyrrolidine-1-carbonyl)benzyl)imidazolidin-2-one (0.030 g, 0.058 mmol, 27%). Mass spectrum (apci) m/z=520.2 (M+H).

The following compounds were prepared according to the method of Example 38.

| | | | MS data |
|---|---|---|---|
| Ex. # | Compound | Name | (apci) m/z |
| 39 | 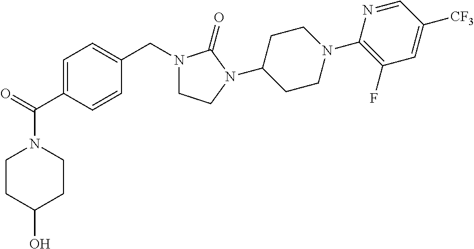 [SMILES: O=C(c1ccc(CN2CCN(C3CCN(c4ncc(C(F)(F)F)cc4F)CC3)C2=O)cc1)N1CCC(O)CC1] | 1-(1-(3-fluoro-5- (trifluoromethyl)pyridin-2- yl)piperidin-4-yl)-3-(4-(4- hydroxypiperidine-1- carbonyl)benzyl) imidazolidin-2-one | 550.2 (M + H) |
| 40 | 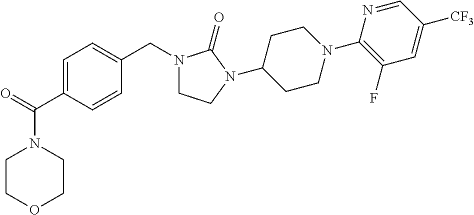 [SMILES: O=C(c1ccc(CN2CCN(C3CCN(c4ncc(C(F)(F)F)cc4F)CC3)C2=O)cc1)N1CCOCC1] | 1-(1-(3-fluoro-5- (trifluoromethyl)pyridin-2- yl)piperidin-4-yl)-3-(4- (morpholine-4- carbonyl)benzyl) imidazolidin-2-one | 536.2 (M + H) |
| 41 | 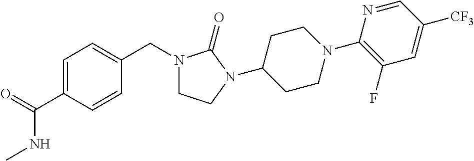 [SMILES: CNC(=O)c1ccc(CN2CCN(C3CCN(c4ncc(C(F)(F)F)cc4F)CC3)C2=O)cc1] | 4-((3-(1-(3-fluoro-5- (trifluoromethyl)pyridin-2- yl)piperidin-4-yl)-2- oxoimidazolidin-1- yl)methyl)-N- methylbenzamide | 480.2 (M + H) |
| 42 | 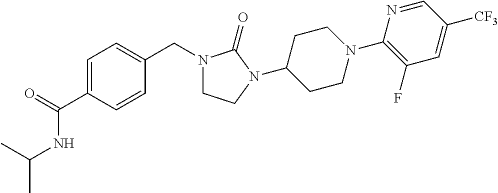 [SMILES: CC(C)NC(=O)c1ccc(CN2CCN(C3CCN(c4ncc(C(F)(F)F)cc4F)CC3)C2=O)cc1] | 4-((3-(1-(3-fluoro-5- (trifluoromethyl)pyridin-2- yl)piperidin-4-yl)-2- oxoimidazolidin-1- yl)methyl)-N- isopropylbenzamide | 508.2 (M + H) |
| 43 | 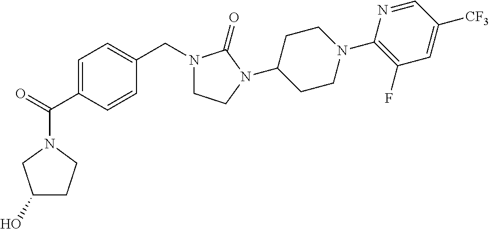 [SMILES: O=C(c1ccc(CN2CCN(C3CCN(c4ncc(C(F)(F)F)cc4F)CC3)C2=O)cc1)N1CC[C@H](O)C1] | (S)-1-(1-(3-fluoro-5- (trifluoromethyl)pyridin-2- yl)piperidin-4-yl)-3-(4-(3- hydroxypyrrolidine-1- carbonyl)benzyl) imidazolidin-2-one | 536.2 (M + H) |
| 44 | 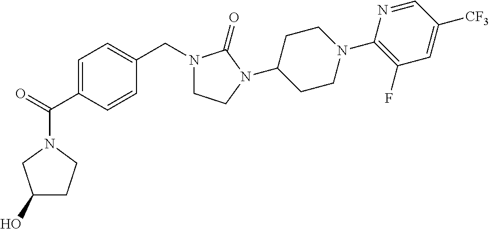 [SMILES: O=C(c1ccc(CN2CCN(C3CCN(c4ncc(C(F)(F)F)cc4F)CC3)C2=O)cc1)N1CC[C@@H](O)C1] | (R)-1-(1-(3-fluoro-5- (trifluoromethyl)pyridin-2- yl)piperidin-4-yl)-3-(4-(3- hydroxypyrrolidine-1- carbonyl)benzyl) imidazolidin-2-one | 536.2 (M + H) |
| 45 | 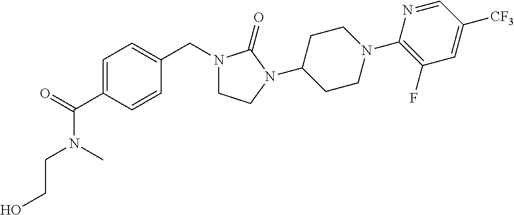 [SMILES: CN(CCO)C(=O)c1ccc(CN2CCN(C3CCN(c4ncc(C(F)(F)F)cc4F)CC3)C2=O)cc1] | 4-((3-(1-(3-fluoro-5- (trifluoromethyl)pyridin-2- yl)piperidin-4-yl)-2- oxoimidazolidin-1- yl)methyl)-N-(2- hydroxyethyl)-N- methylbenzamide | 524.2 (M + H) |

### Example 46

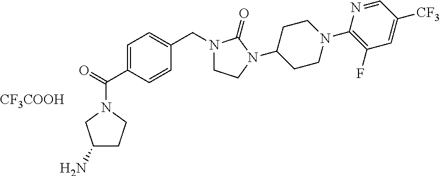 [SMILES: "CF3COOH" ; N[C@H]1CCN(C(=O)c2ccc(CN3CCN(C4CCN(c5ncc(C(F)(F)F)cc5F)CC4)C3=O)cc2)C1]

### (S)-1-(4-(3-aminopyrrolidine-1-carbonyl)benzyl)-3-(1-(3-fluoro-5-(trifluoromethyl)pyridin-2-yl)piperidin-4-yl)imidazolidin-2-one trifluoroacetate

Step 1: 4-((3-(1-(3-fluoro-5-(trifluoromethyl)pyridin-2-yl)piperidin-4-yl)-2-oxoimidazolidin-1-yl)methyl)benzoic acid (Example 38, Step 1; 0.100 g, 0.214 mmol), HATU (0.0897 g, 0.236 mmol), and TEA (0.0448 mL, 0.322 mmol) were dissolved in DMF (1 mL) and the solution was stirred at ambient temperature for 30 minutes. (S)-tert-butyl pyrrolidin-3-ylcarbamate (0.0799 g, 0.429 mmol) was added the mixture stirred for 2 hours. The solution was poured into water (10 mL) and extracted with ethyl acetate. The organic layer was washed with brine, dried over MgSO₄ and filtered. The organic layer was concentrated in vacuo and the residue was purified by column chromatography using 25-75% ethyl acetate/hexane as the eluent to give (S)-tert-butyl 1-(4-((3-(1-(3-fluoro-5-(trifluoromethyl)pyridin-2-yl)piperidin-4-yl)-2-oxoimidazolidin-1-yl)methyl)benzoyl)pyrrolidin-3-ylcarbamate (0.10 g, 76%).

Step 2: A solution of (S)-tert-butyl 1-(4-((3-(1-(3-fluoro-5-(trifluoromethyl)pyridin-2-yl)piperidin-4-yl)-2-oxoimidazolidin-1-yl)methyl)benzoyl)pyrrolidin-3-ylcarbamate (0.10 g, 0.16 mmol) in 50% TFA/DCM (10 mL) was stirred at ambient temperature for 1 hour. The mixture was concentrated in vacuo to give (S)-1-(4-(3-aminopyrrolidine-1-carbonyl)benzyl)-3-(1-(3-fluoro-5-(trifluoromethyl)pyridin-2-yl)piperidin-4-yl)imidazolidin-2-one as the TFA salt (0.107 g, 0.165 mmol, 105%). Mass spectrum (apci) m/z=(535.2, M+H).

### Example 47

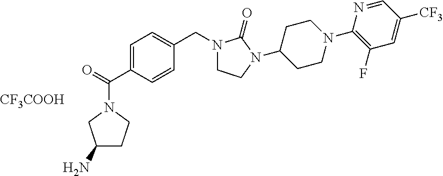 [SMILES: "CF3COOH" ; N[C@@H]1CCN(C(=O)c2ccc(CN3CCN(C4CCN(c5ncc(C(F)(F)F)cc5F)CC4)C3=O)cc2)C1]

### (R)-1-(4-(3-aminopyrrolidine-1-carbonyl)benzyl)-3-(1-(3-fluoro-5-(trifluoromethyl)pyridin-2-yl)piperidin-4-yl)imidazolidin-2-one trifluoroacetate

Prepared according to the method of Example 46. Mass spectrum (apci) m/z=(535.2, M+H).

### Example 48

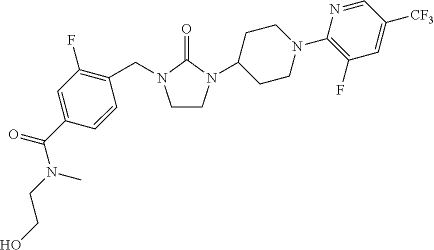 [SMILES: CN(CCO)C(=O)c1ccc(CN2CCN(C3CCN(c4ncc(C(F)(F)F)cc4F)CC3)C2=O)c(F)c1]

### 3-fluoro-4-((3-(1-(3-fluoro-5-(trifluoromethyl)pyridin-2-yl)piperidin-4-yl)-2-oxoimidazolidin-1-yl)methyl)-N-(2-hydroxyethyl)-N-methylbenzamide

Step 1: To a solution of methyl 3-fluoro-4-((2-oxo-3-(piperidin-4-yl)imidazolidin-1-yl)methyl)benzoate 2,2,2-trifluoroacetate (Intermediate 26; 2.4 g, 5.3 mmol) in DMF (20 mL) was added 2,3-difluoro-5-(trifluoromethyl)pyridine (0.98 g, 5.3 mmol) and the reaction was stirred overnight at ambient temperature. The reaction was diluted with EtOAc (200 mL) and the organic layer was washed with HCl (100 mL) and brine (100 mL), dried over MgSO₄, filtered and concentrated in vacuo. The material was purified by column chromatography using 70% EtOAc/Hexane as the eluent to yield methyl 3-fluoro-4-((3-(1-(3-fluoro-5-(trifluoromethyl)pyridin-2-yl)piperidin-4-yl)-2-oxoimidazolidin-1-yl)methyl)benzoate (1.0 g, 2.0 mmol, 38% yield).

Step 2: To a solution of methyl 3-fluoro-4-((3-(1-(3-fluoro-5-(trifluoromethyl)pyridin-2-yl)piperidin-4-yl)-2-oxoimidazolidin-1-yl)methyl)benzoate (1.0 g, 2.0 mmol) in THF was added 1 N lithium hydroxide (20 mL, 20 mmol) and the reaction was stirred for 2 hours at ambient temperature. The reaction was diluted with water (100 mL) and washed with ether (100 mL). The aqueous layer was acidified using 1 N HCl to pH 2 and the aqueous layer was extracted with EtOAc (200 mL). The organic layer was washed with brine (100 mL) and concentrated in vacuo to give 3-fluoro-4-((3-(1-(3-fluoro-5-(trifluoromethyl)pyridin-2-yl)piperidin-4-yl)-2-oxoimidazolidin-1-yl)methyl)benzoic acid (0.60 g, 62%).

Step 3: To a solution of 3-fluoro-4-((3-(1-(3-fluoro-5-(trifluoromethyl)pyridin-2-yl)piperidin-4-yl)-2-oxoimidazolidin-1-yl)methyl)benzoic acid (0.6 g, 1.24 mmol) in DMF (3 mL) was added Hunig's base (0.480 g, 3.72 mmol) and benzotriazol-1-yl-oxytripyrrolidinophosphonium hexafluorophosphate (PyBOP; 0.709 g, 1.36 mmol) and the reaction was stirred for 1 hour at ambient temperature. To the reaction was added 2-(methylamino)ethanol (0.279 g, 3.72 mmol) and the reaction was stirred for 1 hour, diluted with EtOAc (100 mL), washed with 1N HCl (20 mL), 1M NaOH (20 mL) and brine (20 mL), dried over MgSO₄, filtered and concentrated in vacuo. The material was purified by column chromatography using a gradient of 100% EtOAc up to 5% MeOH/DCM in EtOAc as the eluent to provide the crude product as a foam. The crude product was further purified by SP40 reverse preparative HPLC using a gradient of 5% acetonitrile/water up to 80% acetonitrile/water to provide PyBOP as a solid (0.127 g, 0.235 mmol, 18.9% yield). Mass spectrum (apci) m/z=542.2.

### Example 49

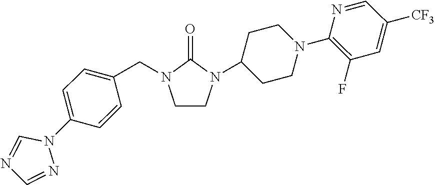 [SMILES: O=C1N(Cc2ccc(-n3cncn3)cc2)CCN1C1CCN(c2ncc(C(F)(F)F)cc2F)CC1]

### 1-(4-(1H-1,2,4-triazol-1-yl)benzyl)-3-(1-(3-fluoro-5-(trifluoromethyl)pyridin-2-yl)piperidin-4-yl)imidazolidin-2-one

Prepared according to the method of Example 1 from Intermediate 27. Mass spectrum (apci) m/z=490.2 (M+H).

### Example 50

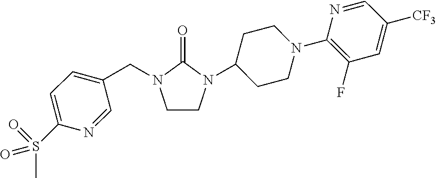 [SMILES: CS(=O)(=O)c1ccc(CN2CCN(C3CCN(c4ncc(C(F)(F)F)cc4F)CC3)C2=O)cn1]

### 1-(1-(3-fluoro-5-(trifluoromethyl)pyridin-2-yl)piperidin-4-yl)-3-((6-(methylsulfonyl)pyridin-3-yl)methyl)imidazolidin-2-one

Prepared according to the method of Example 1 from Intermediate 28. Mass spectrum (apci) m/z=502.1 (M+H).

### Example 51

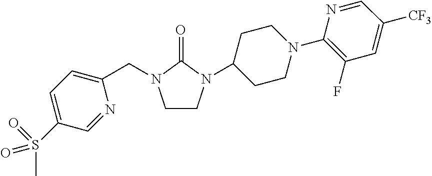 [SMILES: CS(=O)(=O)c1ccc(CN2CCN(C3CCN(c4ncc(C(F)(F)F)cc4F)CC3)C2=O)nc1]

### 1-(1-(3-fluoro-5-(trifluoromethyl)pyridin-2-yl)piperidin-4-yl)-3-((5-(methylsulfonyl)pyridin-2-yl)methyl)imidazolidin-2-one

Prepared according to the method of Example 1 from Intermediate 29. Mass spectrum (apci) m/z=502.1 (M+H).

### Example 52

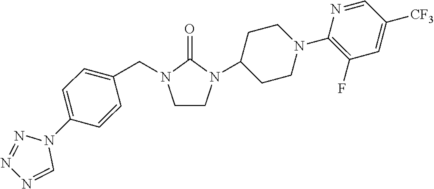 [SMILES: O=C1N(Cc2ccc(-n3cnnn3)cc2)CCN1C1CCN(c2ncc(C(F)(F)F)cc2F)CC1]

### 1-(4-(1H-tetrazol-1-yl)benzyl)-3-(1-(3-fluoro-5-(trifluoromethyl)pyridin-2-yl)piperidin-4-yl)imidazolidin-2-one

Prepared according to the method of Example 1 from Intermediate 30. Mass spectrum (apci) m/z=491.1 (M+H).

1. A compound having the Formula I 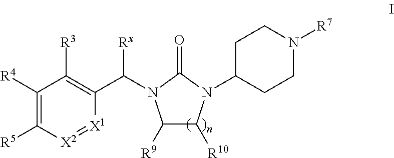
or a pharmaceutically acceptable salt thereof, wherein:
X¹ is N or CR¹ and X² is N or CR², provided that only one of X¹ and X² is N;
R^{x} is H or (1-3C)alkyl;
R¹, R², R³ and R⁴ are independently selected from H, halogen, CF₃, (1-6C)alkyl, CN and (1-6C)alkoxy;
R⁵ is (1-3C alkyl)sulfonyl, (3-6C cycloalkyl)sulfonyl, (cyclopropylmethyl)sulfonyl, phenylsulfonyl, CN, R′R″NHC(═O)—, (1-5C)alkoxyC(═O)— triazolyl, or tetrazolyl optionally substituted with (1-3C)alkyl;
R′ and R″ are independently H or (1-4C)alkyl optionally substituted with OH, or
R′ and R″ together with the atom to which they are attached form a 5-6 membered heterocyclic ring having a ring nitrogen atom and optionally having a second ring heteroatom selected from N and O, wherein said ring is optionally substituted with OH or NH₂;
R⁷ is selected from 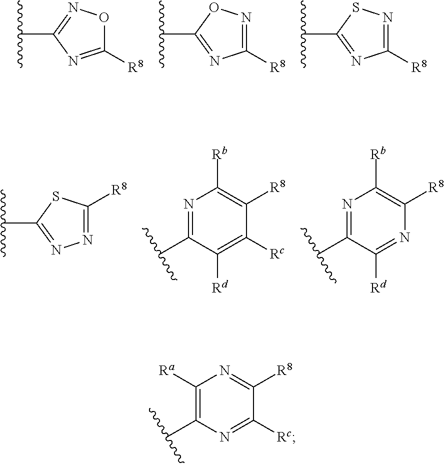
R^{a}, R^{b}, R^{c} and R^{d} are independently H or halogen;
R⁸ is selected from halogen, (1-6C)alkyl, fluoro(1-6C)alkyl, difluoro(1-6C)alkyl, and trifluoro(1-6C)alkyl;
R⁹ is hydrogen or (1-3C)alkyl;
R¹⁰ is hydrogen or (1-3C)alkyl; and
n is 1, 2 or 3, wherein when n is 2 or 3, only one of R¹⁰ can be methyl.

2. (canceled)

3. A compound according to claim 1, where X¹ is CR¹ and X² is CR².

4. A compound according to claim 2, where R¹, R², R³ and R⁴ are independently selected from H and halogen.

5. A compound according to claim 1, where X¹ is N and X² is CR².

6. A compound according to claim 1, where X¹ is CR¹ and X² is N.

7. A compound according to claim 1, wherein R⁵ is (1-3C alkyl)sulfonyl, (3-6C cycloalkyl)sulfonyl, (cyclopropylmethyl)sulfonyl or phenylsulfonyl.

8. A compound according to claim 6, wherein R⁵ is (1-3C alkyl)sulfonyl.

9. (canceled)

10. A compound according to claim 1, wherein R⁵ is R′R″NHC(═O)—.

11. A compound according to claim 1, wherein R⁵ is triazolyl.

12. A compound according to claim 1, wherein R⁵ is tetrazolyl optionally substituted with (1-3C)alkyl.

13. A compound according to claim 1, wherein R⁷ is selected from 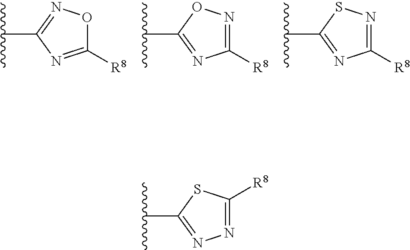
where R⁸ is selected from halogen, (1-6C)alkyl, fluoro(1-6C)alkyl, difluoro(1-6C)alkyl, and trifluoro(1-6C)alkyl.

14-17. (canceled)

18. A compound according to claim 13, wherein R⁸ is selected from (1-6C)alkyl and trifluoro(1-6C)alkyl.

19. A compound according to claim 1, wherein R⁷ is selected from

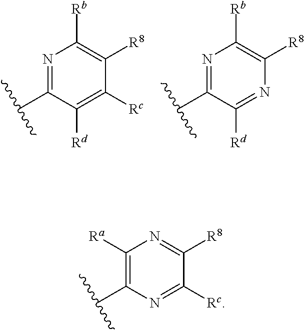

20. A compound according to claim 19, wherein R⁸ is halogen or trifluoro(1-6C)alkyl and R^{a}, R^{b}, R^{c} and R^{d} are H or halogen.

21-25. (canceled)

26. A compound according to claim 1, wherein n is 1.

27. A compound according to claim 1, wherein n is 2.

28. A compound of claim 1, selected from any one of Examples 1-52, or a pharmaceutically acceptable salt thereof.

29. A pharmaceutical composition, which comprises a compound of Formula I as defined in claim 1, or a pharmaceutically acceptable salt thereof, and a pharmaceutically acceptable diluent, carrier or excipient.

30. A method of treating a disease or condition selected from type 2 diabetes, which comprises administering to said mammal a therapeutically effective amount of a compound of Formula I as defined in claim 1 or a pharmaceutically acceptable salt thereof.

31-33. (canceled)

34. A process for the preparation of a compound of Formula I, which comprises:
(a) reacting a corresponding compound of Formula II 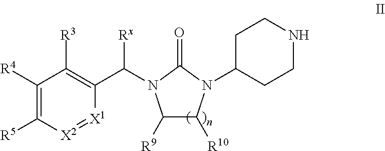
where X¹, X², R³, R⁴, R⁵, R^{x}, R⁹, R¹⁰ and n are as defined for Formula I, with a compound having the formula L¹-R⁷ where L¹ is a leaving atom and R⁷ is as defined for Formula I, in the presence of a base; or
(b) for a compound of Formula I where R⁷ is 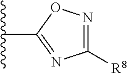
where R⁸ is as defined for Formula I, reacting a corresponding compound of Formula III 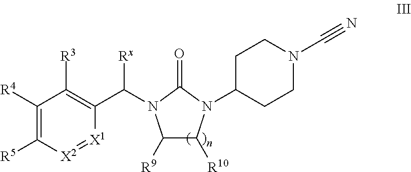
where X¹, X², R³, R⁴, R⁵, R^{x}, R⁹, R¹⁰ and n are as defined for Formula I, with a corresponding compound having the formula 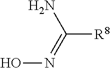
in the presence of a Lewis acid; or
(c) for a compound of Formula I where R⁷ is 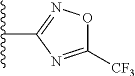 [SMILES: *c1noc(C(F)(F)F)n1]
reacting a compound of Formula III 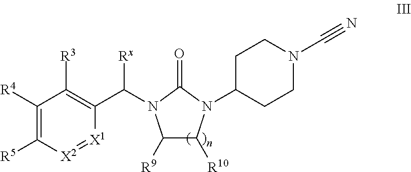
where X¹, X², R³, R⁴, R⁵, R^{x}, R⁹, R¹⁰ and n are as defined for Formula I, with hydroxylamine followed by treatment with 2,2,2-trifluoroacetic anhydride; or
(d) for a compound of Formula I wherein R⁵ is CN, reacting a corresponding compound having the formula IV 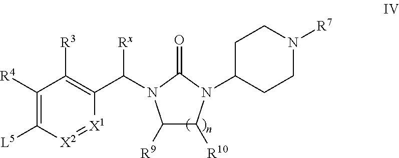
where L⁵ is a leaving group or atom, and X¹, X², R³, R⁴, R⁵, R^{x}, R⁹, R¹⁰ and n are as defined for Formula I, in the presence of a metal catalyst CuCN; or
(e) for a compound of Formula I wherein R⁵ is R′R″NHC(═O)—, and R′ and R″ together with the atom to which they are attached form a 5-6 membered heterocyclic ring having a ring nitrogen atom and optionally having a second ring heteroatom selected from N and O, wherein said ring is optionally substituted with OH or NH₂, reacting a corresponding compound having the formula V 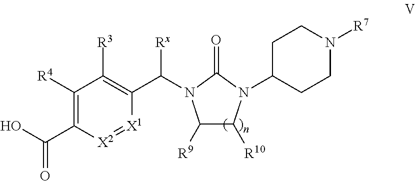
where X¹, X², R³, R⁴, R⁵, R^{x}, R⁹, R¹⁰ and n are as defined for Formula I, with a reagent having the formula 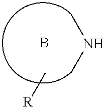
where ring B is a 5-6 membered heterocyclic ring having a ring nitrogen atom and optionally having a second ring heteroatom selected from N and O, wherein said ring is optionally substituted with OP¹ or NHP² where R¹ is hydrogen or a hydroxyl protecting group, and P² is hydrogen or an amino protecting group, in the presence of a coupling reagent; and
optionally removing any protecting groups and optionally preparing a salt thereof.

35. A compound having the Formula II 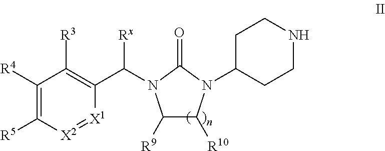
wherein:
R^{x} is H or (1-3C)alkyl;
X¹ is N or CR¹ and X² is N or CR², provided that only one of X¹ and X² is N;
R¹, R², R³ and R⁴ are independently selected from H, halogen, CF₃, (1-6C)alkyl, CN and (1-6C)alkoxy;
R⁵ is (1-3C alkyl)sulfonyl, (3-6C cycloalkyl)sulfonyl, (cyclopropylmethyl)sulfonyl or phenylsulfonyl;
R⁹ is hydrogen or methyl;
R¹⁰ is hydrogen or methyl; and
n is 1, 2 or 3, wherein when n is 2 or 3, only one of R¹⁰ can be methyl, with the proviso that Formula II is not 1-[4-methylsulfphonyl)benzyl]-3-piperidin-4-ylimidazolidin-2-one.

36. A compound having the Formula II-A 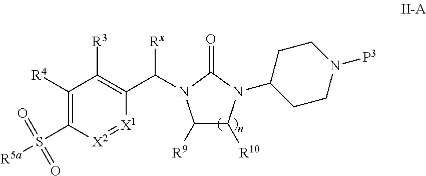
wherein:
P³ is an amine protecting group other than benzyl;
X¹ is N or CR¹ and X² is N or CR², provided that only one of X¹ and X² is N;
R^{x} is H or (1-3C)alkyl;
R¹, R², R³ and R⁴ are independently selected from H, halogen, CF₃, (1-6C)alkyl, CN and (1-6C)alkoxy;
R^{5a} is (1-3C)alkyl, (3-6C)cycloalkyl, cyclopropylmethyl or phenyl;
R⁹ is hydrogen or methyl;
R¹⁰ is hydrogen or methyl; and
n is 1, 2 or 3, wherein when n is 2 or 3, only one of R¹⁰ can be methyl.

37. The compound of claim 36, wherein P³ is t-butoxycarbonyl.
